(19) United States
(12) Patent Application Publication
(10) Pub. No.: US 2018/0334523 A1
(43) Pub. Date: Nov. 22, 2018

(54) NOVOLAC RESIN AND RESIST FILM

(71) Applicant: DIC Corporation, Tokyo (JP)

(72) Inventors: Tomoyuki Imada, Ichihara-shi (JP); Yusuke Sato, Ichihara-shi (JP)

(73) Assignee: DIC Corporation, Tokyo (JP)

(21) Appl. No.: 15/777,739

(22) PCT Filed: Nov. 17, 2016
(86) PCT No.: PCT/JP2016/084055

§ 371 Date: May 21, 2018

(30) Foreign Application Priority Data
Dec. 7, 2015 (JP) 2015-238538

(51) Int. Cl. C08G 8/36; C08G 8/08; C07C 39/15; G03F 7/039

(52) U.S. Cl. CPC C08G 8/36; G03F 7/039; C07C 39/15; C08G 8/08

(57) ABSTRACT

Provided are a novolac resin having developability, heat resistance, and dry etching resistance, and a photosensitive composition, a curable composition, and a resist film. A novolac resin including, as a repeating unit, a structural moiety represented by Structural Formula (1) or (2):

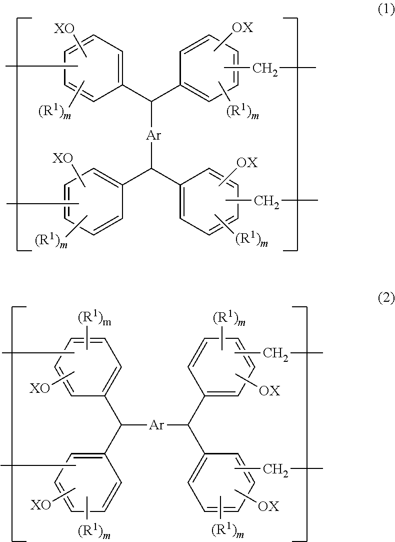

(in the formula, Ar represents an arylene group, R¹'s each independently represent any one of a hydrogen atom, an alkyl group, an alkoxy group, and a halogen atom, m's each independently represent an integer of 1 to 3, and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group) in which at least one of X's present in the resin is any one of a tertiary alkyl group, an alkoxyalkyl group, an acyl group, an alkoxycarbonyl group, a hetero atom-containing cyclic hydrocarbon group, and a trialkylsilyl group.

### TECHNICAL FIELD

The present invention relates to a novolac resin having excellent developability, heat resistance, and dry etching resistance and a resist film formed using the same.

### BACKGROUND ART

A resin containing a phenolic hydroxyl group is used in an adhesive, a molding material, paint, a photoresist material, an epoxy resin raw material, a curing agent for an epoxy resin, and the like. Since the heat resistance and moisture resistance of the cured product of the resin containing a phenolic hydroxyl group are excellent, the resin is also widely used in the electrical and electronic field such as a semiconductor sealing material or an insulating material for a printed wiring board, as a curable composition including the resin containing phenolic hydroxyl group itself as a main agent, a curing agent for an epoxy resin, or the like.

Among these, in the field of a photoresist, a wide variety of resist pattern forming methods subdivided according to the use or the function have been developed one after another. Accordingly, performances required for a resin material for a resist have become more sophisticated and diversified. For example, high developability is required in order to accurately form a fine pattern on a highly-integrated semiconductor with high production efficiency, and in the case of using the resin material in a resist underlayer film, dry etching resistance and heat resistance are required. In the case of using the resin material in a resist permanent film, especially high heat resistance is required.

While the phenolic hydroxyl group-containing resin most widely used for a photoresist is a cresol novolac-type, this type of resin cannot meet the highly sophisticated and diversified performances currently required in the market, and the heat resistance and developability thereof are also not sufficient (refer to PTL 1).

### CITATION LIST

### Patent Literature

[PTL 1] JP-A-2-55359

### SUMMARY OF INVENTION

### Technical Problem

Therefore, an object of the present invention is to provide a novolac resin having excellent developability, heat resistance, and dry etching resistance, and a photosensitive composition, a curable composition, and a resist film, each including the same.

### Solution to Problem

The present inventors have conducted extensive research in order to solve the problem, and as a result, has found that a resin obtained by introducing an acid dissociable protecting group to a ladder-like novolac-type containing resin having a phenolic hydroxyl group, which is obtained by reacting a tetrafunctional phenol compound with formaldehyde, has excellent developability, heat resistance, and dry etching resistance, thus completing the present invention.

That is, the present invention relates to a novolac resin including, as a repeating unit, a structural moiety represented by Structural Formula (1) or (2):

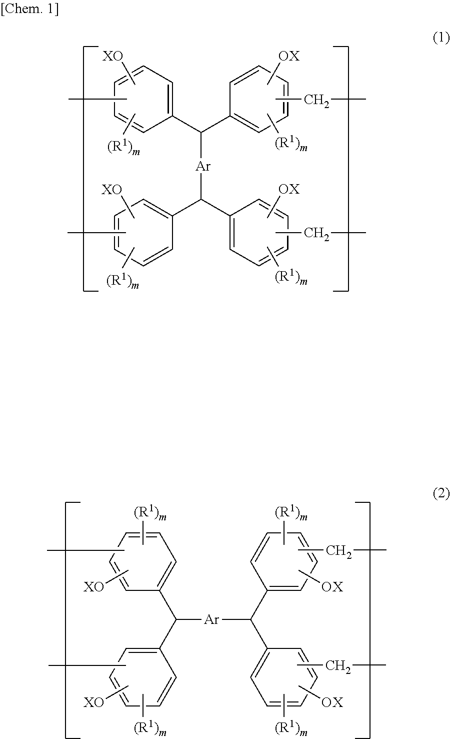

(in the formula, Ar represents an arylene group; R¹'s each independently represent any one of a hydrogen atom, an alkyl group, an alkoxy group, and a halogen atom, and m's each independently represent an integer of 1 to 3; and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group), in which at least one of X's present in the resin is any one of a tertiary alkyl group, an alkoxyalkyl group, an acyl group, an alkoxycarbonyl group, a hetero atom-containing cyclic hydrocarbon group, and a trialkylsilyl group.

The present invention further relates to a photosensitive composition including the novolac resin and a photosensitive agent.

The present invention further relates to a resist film, which is formed from the photosensitive composition.

The present invention further relates to a curable composition including the novolac resin and a curing agent.

The present invention further relates to a resist film, which is formed from the curable composition.

The present invention further relates to a method of producing a novolac resin, including reacting a tetrafunctional phenol compound (A) represented by Structural Formula (4) with formaldehyde as essential components so as to obtain an intermediate novolac resin:

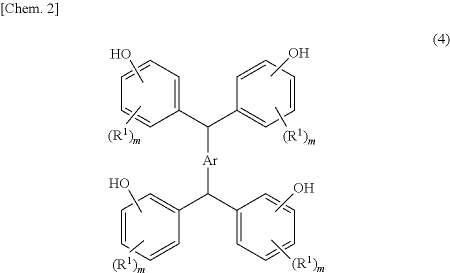

(in the formula, Ar represents an arylene group; R¹'s each independently represent any one of a hydrogen atom, an alkyl group, an alkoxy group, and a halogen atom, and m's each independently represent an integer of 1 to 3); and substituting a portion or all of hydrogen atoms of phenolic hydroxyl groups of the obtained intermediate novolac resin with any one of a tertiary alkyl group, an alkoxyalkyl group, an acyl group, an alkoxycarbonyl group, a hetero atom-containing cyclic hydrocarbon group, and a trialkylsilyl group.

### Advantageous Effects of Invention

According to the present invention, it is possible to provide a novolac resin having excellent developability, heat resistance, and dry etching resistance, and a photosensitive composition, a curable composition, and a resist film, each including the same.

### BRIEF DESCRIPTION OF DRAWINGS

FIG. 1 is a GPC chart diagram of a tetrafunctional phenol compound (A-1) obtained in Production Example 1.

FIG. 2 is a ¹H-NMR chart diagram of the tetrafunctional phenol compound (A-1) obtained in Production Example 1.

FIG. 3 is a GPC chart diagram of an intermediate novolac resin (1) obtained in Production Example 2.

FIG. 4 is a ¹³C-NMR chart diagram of the intermediate novolac resin (1) obtained in Production Example 2.

FIG. 5 is a TOF-MS chart diagram of the intermediate novolac resin (1) obtained in Production Example 2.

FIG. 6 is a GPC chart diagram of an intermediate novolac resin (2) obtained in Production Example 2.

FIG. 7 is a ¹³C-NMR chart diagram of the intermediate novolac resin (2) obtained in Production Example 2.

FIG. 8 is a TOF-MS chart diagram of the intermediate novolac resin (2) obtained in Production Example 2.

### DESCRIPTION OF EMBODIMENTS

Hereinafter, the present invention will be described in detail.

The novolac resin of the present invention includes, as a repeating unit, a structural moiety represented by Structural Formula (1) or (2):

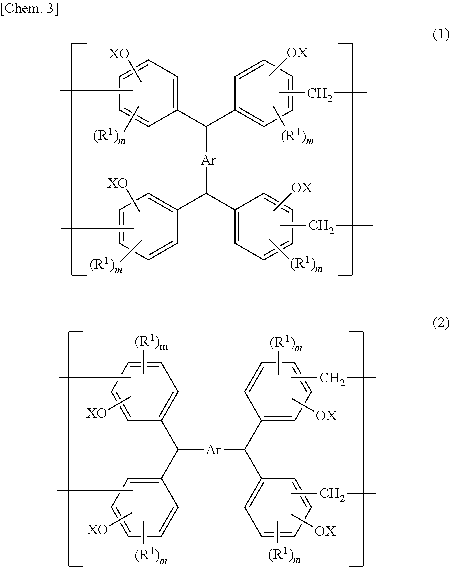

(in the formula, Ar represents an arylene group; R¹'s each independently represent any one of a hydrogen atom, an alkyl group, an alkoxy group, and a halogen atom, and m's each independently represent an integer of 1 to 3; and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group), in which at least one of X's present in the resin is any one of a tertiary alkyl group, an alkoxyalkyl group, an acyl group, an alkoxycarbonyl group, a hetero atom-containing cyclic hydrocarbon group, and a trialkylsilyl group.

The novolac resin of the present invention has a so-called ladder-like rigid and highly symmetrical molecular structure in which the structural moieties represented by Structural Formula (3):

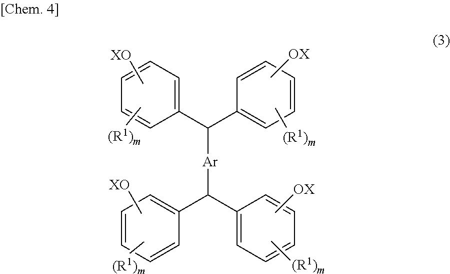

(in the formula, Ar represents an arylene group; R¹'s each independently represent any one of a hydrogen atom, an alkyl group, an alkoxy group, and a halogen atom, and m's each independently represent an integer of 1 to 3; and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group) are knotted with each other by two methylene groups, and thus realizes unprecedented high heat resistance and dry etching resistance.

R¹'s in Structural Formula (1) or (2) each independently represent any one of a hydrogen atom, an alkyl group, an alkoxy group, and a halogen atom. Examples of the alkyl group include a methyl group, an ethyl group, a propyl group, a butyl group, a pentyl group, a hexyl group, and a cyclohexyl group. Examples of the alkoxy group include a methoxy group, an ethoxy group, a propyloxy group, a butoxy group, a pentyloxy group, a hexyloxy group, and a cyclohexyloxy group. Examples of the halogen atom include a fluorine atom, a chlorine atom, and a bromine atom.

Among them, in view of obtaining the novolac resin having excellent balance between the heat resistance and the developability, R¹ is preferably an alkyl group. In view of excellent effect of improving heat resistance or electron donating properties to an aromatic nucleus due to suppression of the molecular motion, and easy industrial availability, R¹ is particularly preferably a methyl group.

In addition, m's in Structural Formula (1) or (2) are each independently an integer of 1 to 3. Among them, in view of obtaining the novolac resin having excellent balance between the heat resistance and the developability, m's are each preferably 1 or 2.

Ar in Structural Formula (1) or (2) is an arylene group, and examples thereof include a phenylene group, a naphthylene group, an anthrylene group, and a structural moiety obtained by substituting one or a plurality of hydrogen atoms of these aromatic nuclei with any one of an alkyl group, an alkoxy group, and a halogen atom. Here, the alkyl group, the alkoxy group, and the halogen atom are those exemplified as R¹. Among these, in view of excellent symmetry of the molecular structure and obtaining the novolac resin having excellent developability, heat resistance, and dry etching resistance, Ar is preferably a phenylene group.

X in Structural Formula (1) or (2) is any one of a hydrogen atom, a tertiary alkyl group, an alkoxyalkyl group, an acyl group, an alkoxycarbonyl group, a hetero atom-containing cyclic hydrocarbon group, and a trialkylsilyl group. Examples of the tertiary alkyl group include a t-butyl group, and a t-pentyl group. Examples of the alkoxyalkyl group include a methoxyethyl group, an ethoxyethyl group, a propoxyethyl group, a butoxyethyl group, a cyclohexyloxyethyl group, and a phenoxyethyl group. Examples of the acyl group include an acetyl group, an ethanoyl group, a propanoyl group, a butanoyl group, a cyclohexanecarbonyl group, and a benzoyl group. Examples of the alkoxycarbonyl group include a methoxycarbonyl group, an ethoxycarbonyl group, a propoxycarbonyl group, a butoxycarbonyl group, a cyclohexyloxycarbonyl group, and a phenoxycarbonyl group. Examples of the hetero atom-containing cyclic hydrocarbon group include a tetrahydrofuranyl group, and a tetrahydropyranyl group. Examples of the trialkylsilyl group include a trimethylsilyl group, a triethylsilyl group, and a t-butyldimethylsilyl group.

Among them, in view of obtaining the novolac resin having excellent photosensitivity, resolution, and alkaline developability, any of an alkoxyalkyl group, an alkoxycarbonyl group, and a hetero atom-containing cyclic hydrocarbon group is preferable, and an ethoxyethyl group or a tetrahydropyranyl group is preferable.

In the novolac resin of the present invention, in the structural moiety (X is any one of a hydrogen atom, a tertiary alkyl group, an alkoxyalkyl group, an acyl group, an alkoxycarbonyl group, a hetero atom-containing cyclic hydrocarbon group, and a trialkylsilyl group) represented by —OX, a proportion of structural moiety (OX′) in which X is any one of a tertiary alkyl group, an alkoxyalkyl group, an acyl group, an alkoxycarbonyl group, a hetero atom-containing cyclic hydrocarbon group, and a trialkylsilyl group is preferably 30% to 100% and is more preferably 70% to 100% in view of obtaining the novolac resin having excellent performance balances between transparency and light transparency, and alkaline developability and resolution.

In the present invention, a presence ratio of the structural moiety (OX′) in which X is any one of a tertiary alkyl group, an alkoxyalkyl group, an acyl group, an alkoxycarbonyl group, a hetero atom-containing cyclic hydrocarbon group, and a trialkylsilyl group is a value calculated from a ratio of a peak of 145 to 160 ppm derived from the structural moiety (OH) in which X is a hydrogen atom, that is, a carbon atom on the benzene ring to which the phenolic hydroxyl group is bonded, to a peak of 95 to 105 ppm derived from the hydrogen atom in X bonded to an oxygen atom derived from a phenolic hydroxyl group in the structural moiety (OX′) in which X is any one of a tertiary alkyl group, an alkoxyalkyl group, an acyl group, an alkoxycarbonyl group, a hetero atom-containing cyclic hydrocarbon group, and a trialkylsilyl group, in ¹³C-NMR measurement measured under the following conditions.

Apparatus: “JNM-LA300” manufactured by JEOL Ltd.

Solvent: DMSO-d₆

A method of producing the novolac resin of the present invention is not particularly limited, and examples thereof include a method including reacting a tetrafunctional phenol compound (A) represented by Structural Formula (4) with formaldehyde as essential components so as to obtain an intermediate novolac resin:

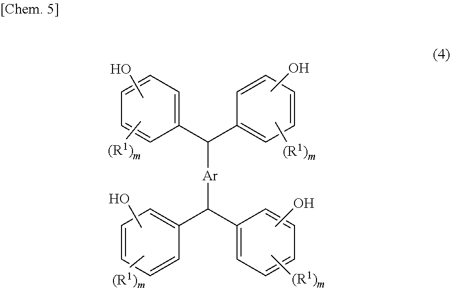

(in the formula, Ar represents an arylene group; R¹'s each independently represent any one of a hydrogen atom, an alkyl group, an alkoxy group, and a halogen atom, and m's each independently represent an integer of 1 to 3); and substituting a portion or all of hydrogen atoms of phenolic hydroxyl groups of the obtained intermediate novolac resin with any one of a tertiary alkyl group, an alkoxyalkyl group, an acyl group, an alkoxycarbonyl group, a hetero atom-containing cyclic hydrocarbon group, and a trialkylsilyl group.

R¹ in Structural Formula (4) is the same as R¹ in Structural Formula (1) or (2), and specific examples of the tetrafunctional phenol compound (A) represented by Structural Formula (4) include those having a molecular structure represented by any one of Structural Formulae (4-1) to (4-45).

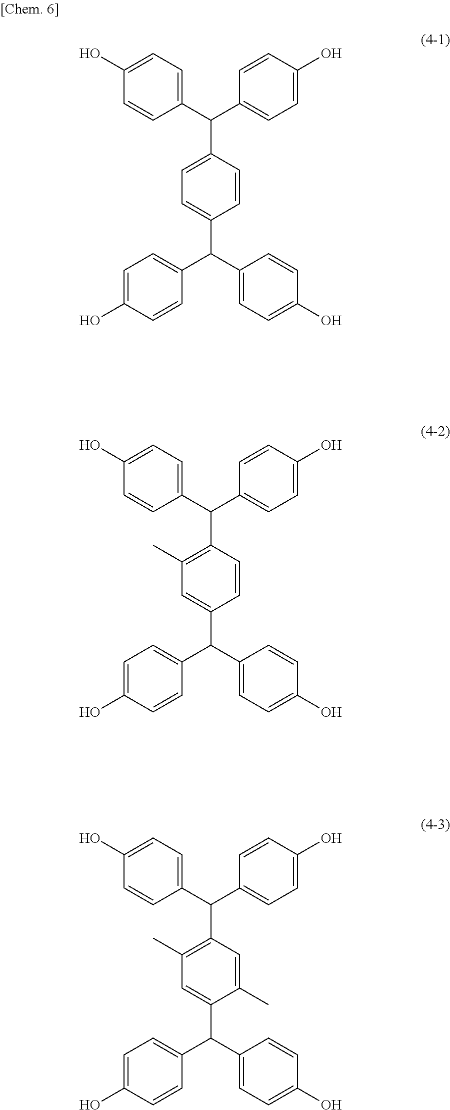 [SMILES: Oc1ccc(C(c2ccc(O)cc2)c2ccc(C(c3ccc(O)cc3)c3ccc(O)cc3)cc2)cc1 ; Cc1cc(C(c2ccc(O)cc2)c2ccc(O)cc2)ccc1C(c1ccc(O)cc1)c1ccc(O)cc1 ; Cc1cc(C(c2ccc(O)cc2)c2ccc(O)cc2)c(C)cc1C(c1ccc(O)cc1)c1ccc(O)cc1]

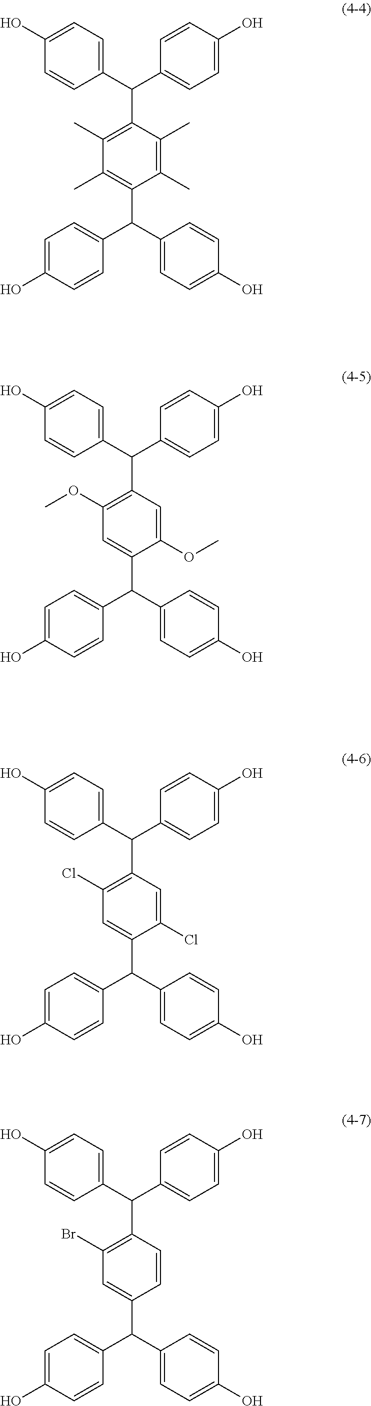 [SMILES: Cc1c(C)c(C(c2ccc(O)cc2)c2ccc(O)cc2)c(C)c(C)c1C(c1ccc(O)cc1)c1ccc(O)cc1 ; COc1cc(C(c2ccc(O)cc2)c2ccc(O)cc2)c(OC)cc1C(c1ccc(O)cc1)c1ccc(O)cc1 ; Oc1ccc(C(c2ccc(O)cc2)c2cc(Cl)c(C(c3ccc(O)cc3)c3ccc(O)cc3)cc2Cl)cc1 ; Oc1ccc(C(c2ccc(O)cc2)c2ccc(C(c3ccc(O)cc3)c3ccc(O)cc3)c(Br)c2)cc1]

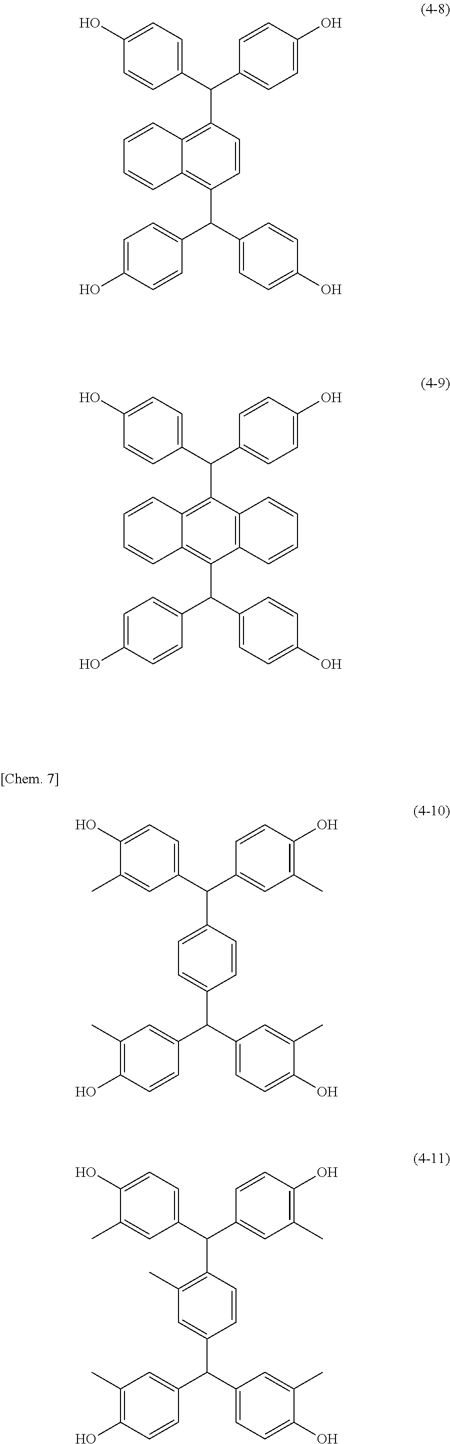 [SMILES: Oc1ccc(C(c2ccc(O)cc2)c2ccc(C(c3ccc(O)cc3)c3ccc(O)cc3)c3ccccc23)cc1 ; Oc1ccc(C(c2ccc(O)cc2)c2c3ccccc3c(C(c3ccc(O)cc3)c3ccc(O)cc3)c3ccccc23)cc1 ; Cc1cc(C(c2ccc(C(c3ccc(O)c(C)c3)c3ccc(O)c(C)c3)cc2)c2ccc(O)c(C)c2)ccc1O ; Cc1cc(C(c2ccc(O)c(C)c2)c2ccc(C(c3ccc(O)c(C)c3)c3ccc(O)c(C)c3)c(C)c2)ccc1O]

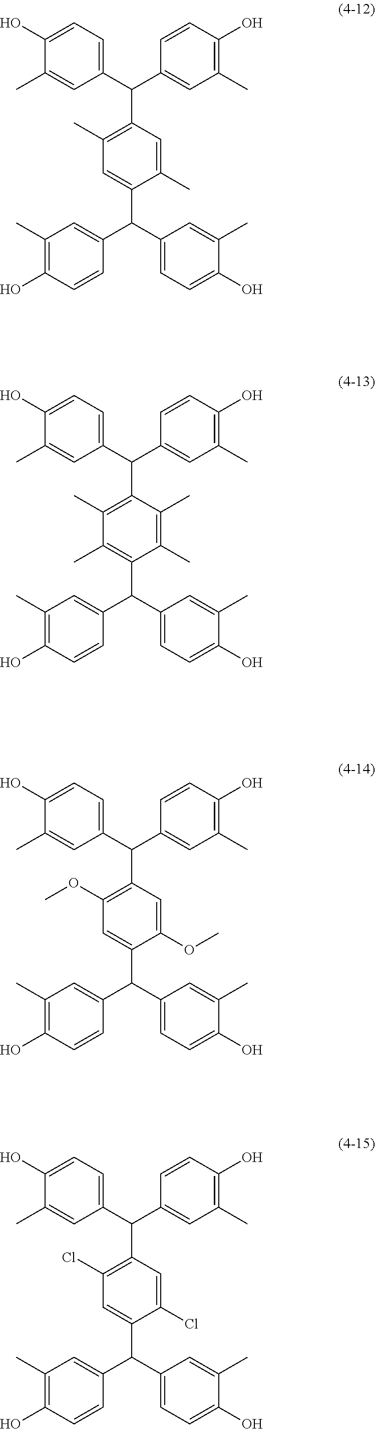 [SMILES: Cc1cc(C(c2ccc(O)c(C)c2)c2cc(C)c(C(c3ccc(O)c(C)c3)c3ccc(O)c(C)c3)cc2C)ccc1O ; Cc1cc(C(c2ccc(O)c(C)c2)c2c(C)c(C)c(C(c3ccc(O)c(C)c3)c3ccc(O)c(C)c3)c(C)c2C)ccc1O ; COc1cc(C(c2ccc(O)c(C)c2)c2ccc(O)c(C)c2)c(OC)cc1C(c1ccc(O)c(C)c1)c1ccc(O)c(C)c1 ; Cc1cc(C(c2ccc(O)c(C)c2)c2cc(Cl)c(C(c3ccc(O)c(C)c3)c3ccc(O)c(C)c3)cc2Cl)ccc1O]

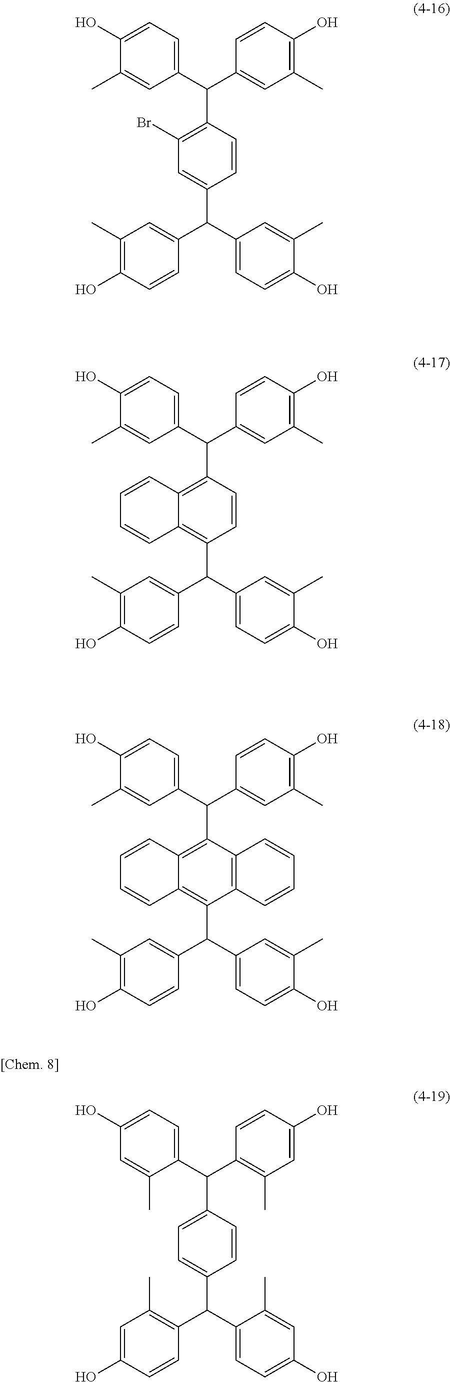 [SMILES: Cc1cc(C(c2ccc(O)c(C)c2)c2ccc(C(c3ccc(O)c(C)c3)c3ccc(O)c(C)c3)c(Br)c2)ccc1O ; Cc1cc(C(c2ccc(O)c(C)c2)c2ccc(C(c3ccc(O)c(C)c3)c3ccc(O)c(C)c3)c3ccccc23)ccc1O ; Cc1cc(C(c2ccc(O)c(C)c2)c2c3ccccc3c(C(c3ccc(O)c(C)c3)c3ccc(O)c(C)c3)c3ccccc23)ccc1O ; Cc1cc(O)ccc1C(c1ccc(C(c2ccc(O)cc2C)c2ccc(O)cc2C)cc1)c1ccc(O)cc1C]

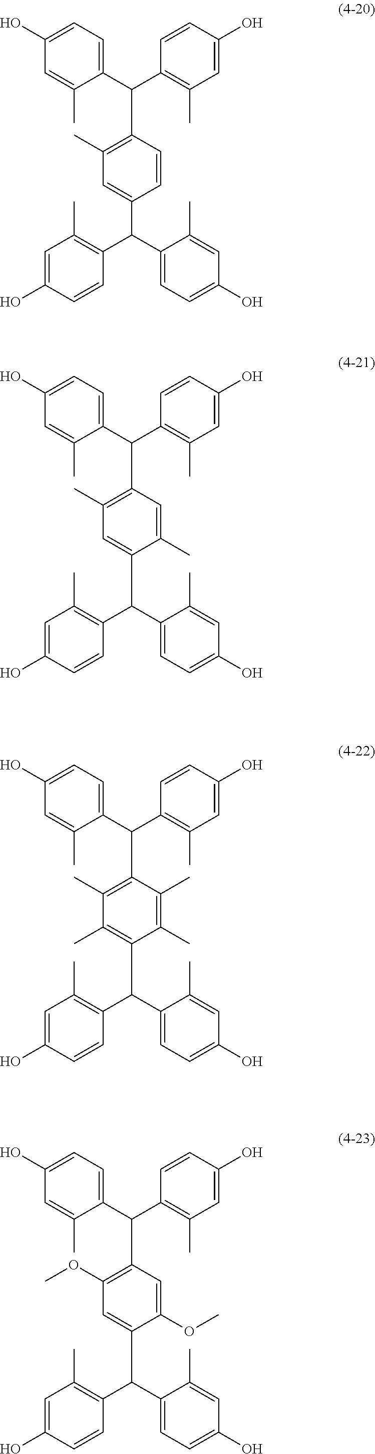 [SMILES: Cc1cc(O)ccc1C(c1ccc(C(c2ccc(O)cc2C)c2ccc(O)cc2C)c(C)c1)c1ccc(O)cc1C ; Cc1cc(O)ccc1C(c1ccc(O)cc1C)c1cc(C)c(C(c2ccc(O)cc2C)c2ccc(O)cc2C)cc1C ; Cc1cc(O)ccc1C(c1ccc(O)cc1C)c1c(C)c(C)c(C(c2ccc(O)cc2C)c2ccc(O)cc2C)c(C)c1C ; COc1cc(C(c2ccc(O)cc2C)c2ccc(O)cc2C)c(OC)cc1C(c1ccc(O)cc1C)c1ccc(O)cc1C]

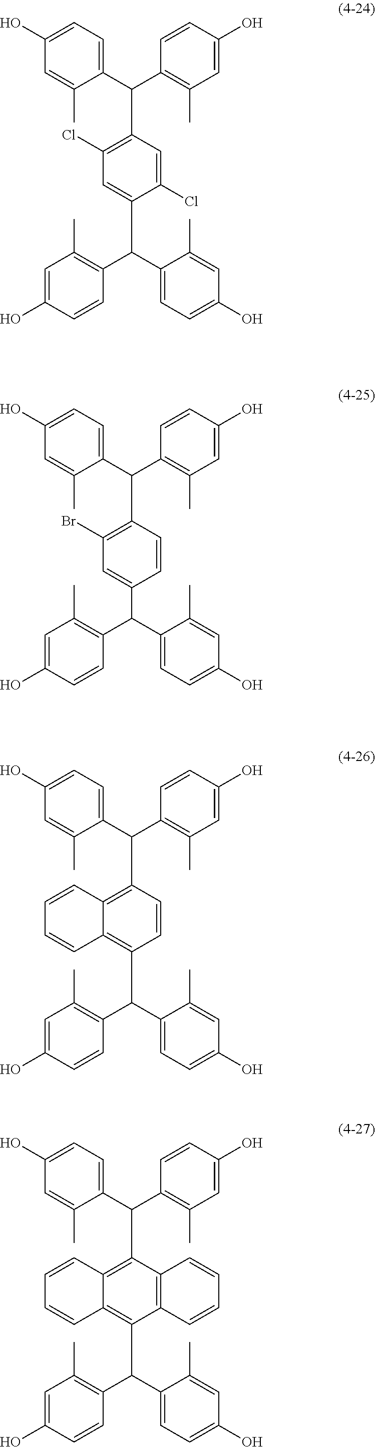 [SMILES: Cc1cc(O)ccc1C(c1ccc(O)cc1C)c1cc(Cl)c(C(c2ccc(O)cc2C)c2ccc(O)cc2C)cc1Cl ; Cc1cc(O)ccc1C(c1ccc(C(c2ccc(O)cc2C)c2ccc(O)cc2C)c(Br)c1)c1ccc(O)cc1C ; Cc1cc(O)ccc1C(c1ccc(O)cc1C)c1ccc(C(c2ccc(O)cc2C)c2ccc(O)cc2C)c2ccccc12 ; Cc1cc(O)ccc1C(c1ccc(O)cc1C)c1c2ccccc2c(C(c2ccc(O)cc2C)c2ccc(O)cc2C)c2ccccc12]

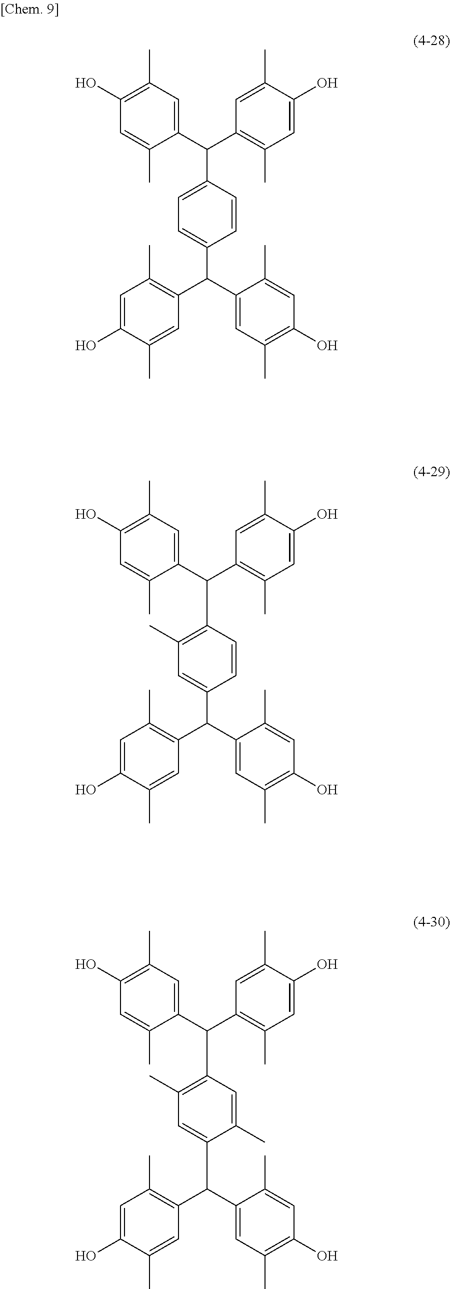 [SMILES: Cc1cc(C(c2ccc(C(c3cc(C)c(O)cc3C)c3cc(C)c(O)cc3C)cc2)c2cc(C)c(O)cc2C)c(C)cc1O ; Cc1cc(C(c2ccc(C(c3cc(C)c(O)cc3C)c3cc(C)c(O)cc3C)c(C)c2)c2cc(C)c(O)cc2C)c(C)cc1O ; Cc1cc(C(c2cc(C)c(O)cc2C)c2cc(C)c(C(c3cc(C)c(O)cc3C)c3cc(C)c(O)cc3C)cc2C)c(C)cc1O]

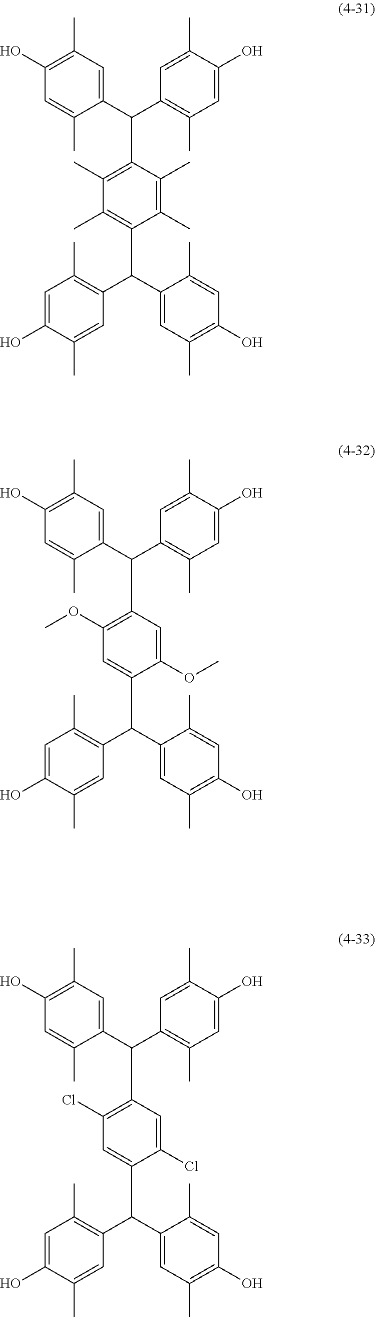 [SMILES: Cc1cc(C(c2cc(C)c(O)cc2C)c2c(C)c(C)c(C(c3cc(C)c(O)cc3C)c3cc(C)c(O)cc3C)c(C)c2C)c(C)cc1O ; COc1cc(C(c2cc(C)c(O)cc2C)c2cc(C)c(O)cc2C)c(OC)cc1C(c1cc(C)c(O)cc1C)c1cc(C)c(O)cc1C ; Cc1cc(C(c2cc(C)c(O)cc2C)c2cc(Cl)c(C(c3cc(C)c(O)cc3C)c3cc(C)c(O)cc3C)cc2Cl)c(C)cc1O]

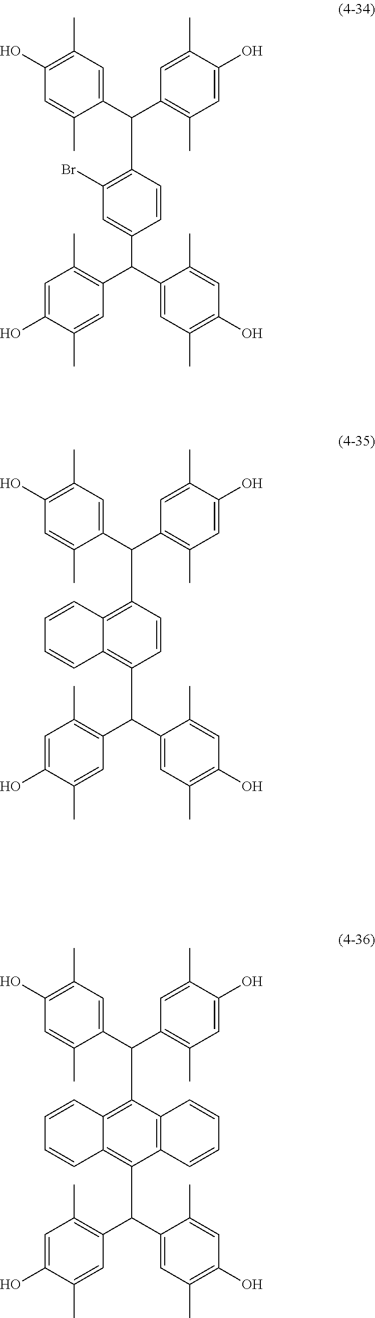 [SMILES: Cc1cc(C(c2ccc(C(c3cc(C)c(O)cc3C)c3cc(C)c(O)cc3C)c(Br)c2)c2cc(C)c(O)cc2C)c(C)cc1O ; Cc1cc(C(c2cc(C)c(O)cc2C)c2ccc(C(c3cc(C)c(O)cc3C)c3cc(C)c(O)cc3C)c3ccccc23)c(C)cc1O ; Cc1cc(C(c2cc(C)c(O)cc2C)c2c3ccccc3c(C(c3cc(C)c(O)cc3C)c3cc(C)c(O)cc3C)c3ccccc23)c(C)cc1O]

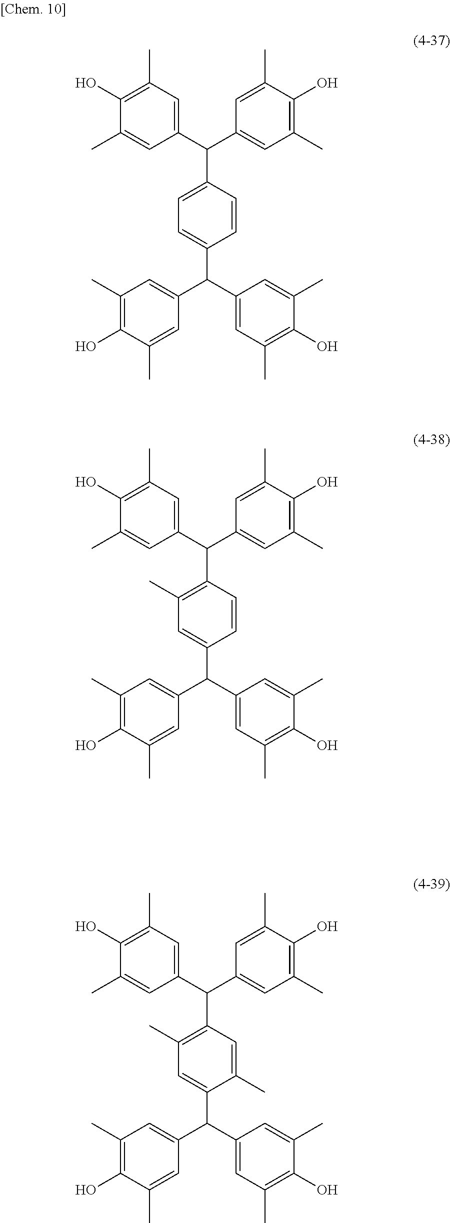 [SMILES: Cc1cc(C(c2ccc(C(c3cc(C)c(O)c(C)c3)c3cc(C)c(O)c(C)c3)cc2)c2cc(C)c(O)c(C)c2)cc(C)c1O ; Cc1cc(C(c2cc(C)c(O)c(C)c2)c2cc(C)c(O)c(C)c2)ccc1C(c1cc(C)c(O)c(C)c1)c1cc(C)c(O)c(C)c1 ; Cc1cc(C(c2cc(C)c(O)c(C)c2)c2cc(C)c(O)c(C)c2)c(C)cc1C(c1cc(C)c(O)c(C)c1)c1cc(C)c(O)c(C)c1]

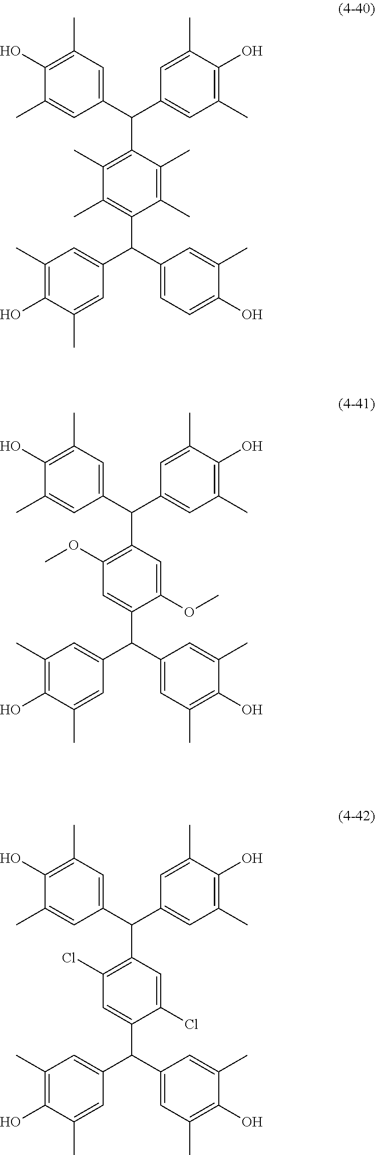 [SMILES: Cc1cc(C(c2cc(C)c(O)c(C)c2)c2c(C)c(C)c(C(c3cc(C)c(O)c(C)c3)c3cc(C)c(O)c(C)c3)c(C)c2C)ccc1O ; COc1cc(C(c2cc(C)c(O)c(C)c2)c2cc(C)c(O)c(C)c2)c(OC)cc1C(c1cc(C)c(O)c(C)c1)c1cc(C)c(O)c(C)c1 ; Cc1cc(C(c2cc(C)c(O)c(C)c2)c2cc(Cl)c(C(c3cc(C)c(O)c(C)c3)c3cc(C)c(O)c(C)c3)cc2Cl)cc(C)c1O]

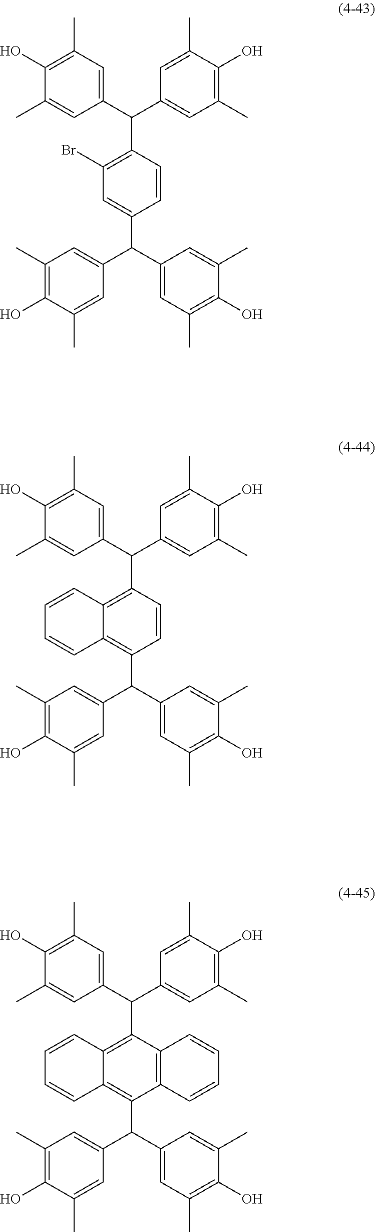 [SMILES: Cc1cc(C(c2cc(C)c(O)c(C)c2)c2ccc(C(c3cc(C)c(O)c(C)c3)c3cc(C)c(O)c(C)c3)c(Br)c2)cc(C)c1O ; Cc1cc(C(c2cc(C)c(O)c(C)c2)c2ccc(C(c3cc(C)c(O)c(C)c3)c3cc(C)c(O)c(C)c3)c3ccccc23)cc(C)c1O ; Cc1cc(C(c2cc(C)c(O)c(C)c2)c2c3ccccc3c(C(c3cc(C)c(O)c(C)c3)c3cc(C)c(O)c(C)c3)c3ccccc23)cc(C)c1O]

The tetrafunctional phenol compound (A) can be obtained by, for example, using a method of reacting the phenol compound (a1) with the aromatic dialdehyde (a2) under the presence of acid catalyst.

The phenol compound (a1) is a compound in which a part or all of the hydrogen atoms bonded to the aromatic rings of phenols are substituted with any one of an alkyl group, an alkoxy group, an aryl group, an aralkyl group, and a halogen atom. Examples of the alkyl group include a methyl group, an ethyl group, a propyl group, a butyl group, a pentyl group, a hexyl group, and a cyclohexyl group. Examples of the alkoxy group include a methoxy group, an ethoxy group, a propyloxy group, a butoxy group, a pentyloxy group, a hexyloxy group, and a cyclohexyloxy group. Examples of the aryl group include a phenyl group, a hydroxyphenyl group, a dihydroxyphenyl group, a hydroxyalkoxyphenyl group, an alkoxyphenyl group, a tolyl group, a xylyl group, a naphthyl group, a hydroxynaphthyl group, and a dihydroxynaphthyl group. Examples of the aralkyl group include a phenylmethyl group, a hydroxyphenyl methyl group, a dihydroxyphenyl methyl group, a tolylmethyl group, a xylylmethyl group, a naphthyl methyl group, a hydroxynaphthyl methyl group, a dihydroxynaphthyl methyl group, a phenylethyl group, a hydroxyphenyl ethyl group, a dihydroxyphenyl ethyl group, a tolylethyl group, a xylylethyl group, a naphthyl ethyl group, a hydroxynaphthyl ethyl group, and a dihydroxynaphthyl ethyl group. Examples of the halogen atom include a fluorine atom, a chlorine atom, and a bromine atom. These phenol compounds may be used singly, or two or more kinds thereof may be used in combination.

Among them, in view of obtaining the novolac resin having excellent developability, heat resistance, and dry etching resistance, alkyl-substituted phenols are preferable, and specifically, o-cresol, m-cresol, p-cresol, 2,5-xylenol, 3,5-xylenol, 3,4-xylenol, 2,4-xylenol, 2,6-xylenol, 2,3,5-trimethyl phenol, and 2,3,6-trimethyl phenol are preferable. Among them, 2,5-xylenol and 2,6-xylenol are particularly preferable.

The aromatic dialdehyde (a2) may be any compound as long as it is a compound obtained by substituting two of the hydrogen atoms bonded to the aromatic ring of an aromatic compound such as benzene, naphthalene, anthracene and a derivative thereof with formyl groups. Among these, in view of excellent symmetry of the molecular structure and obtaining the novolac resin having excellent developability, heat resistance, and dry etching resistance, it is preferable that the compound has a structure in which the two formyl groups are bonded to the aromatic ring at para positions to each other. Examples of such compound include a phenylene-type dialdehyde compound such as terephthalaldehyde, 2-methyl terephthalaldehyde, 2,5-dimethyl terephthalaldehyde, 2,3,5,6-tetramethyl benzene-1,4-dicarbaldehyde, 2,5-dimethoxyterephthalaldehyde, 2,5-dichloroterephthalaldehyde, and 2-bromoterephthalaldehyde; a naphthylene-type dialdehyde compound such as 1,4-naphthalenedicarbaldehyde; and an anthrylene-type dialdehyde compound such as 9,10-anthracenedicarbaldehyde. Each of these compounds may be used singly, or two or more kinds thereof may be used in combination.

Among these aromatic dialdehydes (a2), in view of excellent symmetry of the molecular structure and obtaining the novolac resin having excellent developability, heat resistance, and dry etching resistance, a phenylene-type dialdehyde compound is preferable.

The reaction molar ratio [(a1)/(a2)] of the phenol compound (a1) to the aromatic dialdehyde (a2) is preferably 1/0.1 to 1/0.25, in view of obtaining the desired tetrafunctional phenol compound (A) at a high yield and high purity.

Examples of the acid catalyst used in the reaction between the phenol compound (a1) and the aromatic dialdehyde (a2) include acetic acid, oxalic acid, sulfuric acid, hydrochloric acid, phenolsulfonic acid, p-toluenesulfonic acid, zinc acetate, and manganese acetate. Each of these acid catalysts may be used singly, or two or more kinds thereof may be used in combination. Among these, from the viewpoint of excellent catalytic activity, sulfuric acid and p-toluenesulfonic acid are preferable.

The reaction between the phenol compound (a1) and the aromatic dialdehyde (a2) may be carried out in an organic solvent as necessary. Examples of the solvent used here include a monoalcohol such as methanol, ethanol, and propanol; a polyol such as ethylene glycol, 1,2-propanediol, 1,3-propanediol, 1,4-butanediol, 1,5-pentanediol, 1,6-hexanediol, 1,7-heptanediol, 1,8-octanediol, 1,9-nonanediol, trimethylene glycol, diethylene glycol, polyethylene glycol, and glycerin; a glycol ether such as 2-ethoxyethanol, ethylene glycol monomethyl ether, ethylene glycol monoethyl ether, ethylene glycol monopropyl ether, ethylene glycol monobutyl ether, ethylene glycol monopentyl ether, ethylene glycol dimethyl ether, ethylene glycol ethyl methyl ether, and ethylene glycol monophenyl ether; a cyclic ether such as 1,3-dioxane, 1,4-dioxane, and tetrahydrofuran; a glycol ester such as ethylene glycol acetate; a ketone such as acetone, methyl ethyl ketone, and methyl isobutyl ketone; and aromatic hydrocarbons such as benzene, toluene and xylene. Each of these solvents may be used singly, or two or more kinds thereof may be used in combination. Among these, 2-ethoxyethanol is preferable in view of the excellent solubility of the obtained tetrafunctional phenol compound (A).

The reaction between the phenol compound (a1) and the aromatic dialdehyde (a2) may be carried out, for example, at 60° C. to 140° C. for 0.5 to 100 hours.

After the end of the reaction, the unreacted phenol compound (a1) or the aromatic dialdehyde (a2) and the acid catalyst used are removed from the reaction product, for example, by the method in which the reaction product is put into a poor solvent (S1) for the tetrafunctional phenol compound (A), a precipitate is isolated by filtration, and then the obtained precipitate is redissolved in a solvent (S2) of which solubility for the tetrafunctional phenol compound (A) is high and which is miscible with the poor solvent (S1), so as to obtain the purified tetrafunctional phenol compound (A).

In view of obtaining the novolac resin having both excellent developability and excellent heat resistance, the purity of the tetrafunctional phenol compound (A) calculated from the GPC chart diagram is preferably 90% or higher, more preferably 94% or higher, and particularly preferably 98% or higher. The purity of the tetrafunctional phenol compound (A) can be obtained from the area ratio of the gel permeation chromatography (GPC) chart diagram.

The measurement condition for GPC in the present invention is as follows.

### [Measurement Condition for GPC]

Measuring device: “HLC-8220 GPC” manufactured by TOSOH CORPORATION

Column: “Shodex KF802” (8.0 mmΦ×300 mm) manufactured by SHOWA DENKO K.K.

+“Shodex KF802” (8.0 mmΦ×300 mm) manufactured by SHOWA DENKO K.K.

+“Shodex KF803” (8.0 mmΦ×300 mm) manufactured by SHOWA DENKO K.K.

+“Shodex KF804” (8.0 mmΦ×300 mm) manufactured by SHOWA DENKO K.K.

Column temperature: 40° C.

Detector: RI (differential refractometer)

Data processing: “GPC-8020 MODEL II VERSION 4.30” manufactured by TOSOH CORPORATION

Eluent: tetrahydrofuran

Flow rate: 1.0 ml/min

Sample: a sample obtained by filtering 0.5% by mass (in terms of a resin solid content) of tetrahydrofuran solution through a microfilter

Injection volume: 0.1 ml

Standard sample: the following monodisperse polystyrene (Standard sample: monodisperse polystyrene)

“A-500” manufactured by TOSOH CORPORATION

“A-2500” manufactured by TOSOH CORPORATION

“A-5000” manufactured by TOSOH CORPORATION

“F-1” manufactured by TOSOH CORPORATION

“F-2” manufactured by TOSOH CORPORATION

“F-4” manufactured by TOSOH CORPORATION

“F-10” manufactured by TOSOH CORPORATION

“F-20” manufactured by TOSOH CORPORATION

Examples of the poor solvent (S1) used for the purification of the tetrafunctional phenol compound (A) include water; a monoalcohol such as methanol, ethanol, propanol, and ethoxyethanol; an aliphatic hydrocarbon such as n-hexane, n-heptane, n-octane, and cyclohexane; and an aromatic hydrocarbon such as toluene and xylene. Each of these may be used alone, or two or more kinds thereof may be used in combination. Among these, in view of excellent solubility of the acid catalyst, water, methanol, and ethoxyethanol are preferable.

Examples of the solvent (S2) include a monoalcohol such as methanol, ethanol, and propanol; a polyol such as ethylene glycol, 1,2-propanediol, 1,3-propanediol, 1,4-butanediol, 1,5-pentanediol, 1,6-hexanediol, 1,7-heptanediol, 1,8-octanediol, 1,9-nonanediol, trimethylene glycol, diethylene glycol, polyethylene glycol, and glycerin; a glycol ether such as 2-ethoxyethanol, ethylene glycol monomethyl ether, ethylene glycol monoethyl ether, ethylene glycol monopropyl ether, ethylene glycol monobutyl ether, ethylene glycol monopentyl ether, ethylene glycol dimethyl ether, ethylene glycol ethyl methyl ether, and ethylene glycol monophenyl ether; a cyclic ether such as 1,3-dioxane and 1,4-dioxane; a glycol ester such as ethylene glycol acetate; and a ketone such as acetone, methyl ethyl ketone, and methyl isobutyl ketone. Each of these may be used alone, or two or more kinds thereof may be used in combination. Among these, in the case where water or a monoalcohol is used as the poor solvent (S1), it is preferable that acetone is used as the solvent (S2).

Next, in a step of obtaining the intermediate novolac resin by reacting the tetrafunctional phenol compound (A) with the formaldehyde, the formaldehyde to be used may be formaldehyde in any state, such as formalin that is in the state of an aqueous solution or paraformaldehyde that is in the state of a solid.

In view of the ability to inhibit the resin from having excessively high molecular weight (gelation) and obtaining the novolac resin having suitable molecular weight as a resist material, the reaction ratio of the formaldehyde to the tetrafunctional phenol compound (A) is preferably 0.5 to 7.0 mol and is more preferably 0.6 to 6.0 mol, with respect to 1 mol of the tetrafunctional phenol compound (A).

The reaction between the tetrafunctional phenol compound (A) and formaldehyde is carried out under a conventional acid catalyst condition, as in the general method of producing a novolac resin. Examples of the acid catalyst used here include acetic acid, oxalic acid, sulfuric acid, hydrochloric acid, phenolsulfonic acid, p-toluenesulfonic acid, zinc acetate, and manganese acetate. Each of these acid catalysts may be used singly, or two or more kinds thereof may be used in combination. Among these, from the viewpoint of excellent catalytic activity, sulfuric acid and p-toluenesulfonic acid are preferable.

The reaction between the tetrafunctional phenol compound (A) and the formaldehyde may be carried out in an organic solvent as necessary. Examples of the solvent used here include a monoalcohol such as methanol, ethanol, and propanol; monocarboxylic acid such as acetic acid, propionic acid, butyric acid, pentanoic acid, and hexanoic acid; a polyol such as ethylene glycol, 1,2-propanediol, 1,3-propanediol, 1,4-butanediol, 1,5-pentanediol, 1,6-hexanediol, 1,7-heptanediol, 1,8-octanediol, 1,9-nonanediol, trimethylene glycol, diethylene glycol, polyethylene glycol, and glycerin; a glycol ether such as 2-ethoxyethanol, ethylene glycol monomethyl ether, ethylene glycol monoethyl ether, ethylene glycol monopropyl ether, ethylene glycol monobutyl ether, ethylene glycol monopentyl ether, ethylene glycol dimethyl ether, ethylene glycol ethyl methyl ether, and ethylene glycol monophenyl ether; a cyclic ether such as 1,3-dioxane, 1,4-dioxane, and tetrahydrofuran; a glycol ester such as ethylene glycol acetate; and a ketone such as acetone, methyl ethyl ketone, and methyl isobutyl ketone. Each of these solvents may be used singly, or two or more kinds thereof may be used in combination. Among these, from the viewpoint of excellent solubility of novolac resin to be obtained, a mixed solvent of a monoalcohol such as methanol and monocarboxylic acid such as acetic acid is preferable.

The reaction between the tetrafunctional phenol compound (A) and the formaldehyde may be carried out, for example, at 60° C. to 140° C. for 0.5 to 100 hours. After the end of the reaction, an operation such as a reprecipitation operation which is performed by adding water to the reaction product is carried out, so as to obtain an intermediate novolac resin.

The weight average molecular weight (Mw) of the intermediate novolac resin is preferably 1,500 to 30,000 in view of obtaining the novolac resin which is a final objective and is excellent in the heat resistance, the photosensitivity, and the alkali developability. The polydispersity (Mw/Mn) of the intermediate novolac resin is preferably 1 to 10 in view of obtaining the novolac resin which is a final objective and is excellent in the heat resistance, the photosensitivity, and the alkali developability. The weight average molecular weight (Mw) and the polydispersity (Mw/Mn) in the present invention are values measured by GPC under the same conditions as the conditions for the calculation of the purity of the tetrafunctional phenol compound (A).

Next, specific examples of the method of substituting a portion or all of hydrogen atoms of phenolic hydroxyl groups of the obtained intermediate novolac resin with any one of a tertiary alkyl group, an alkoxyalkyl group, an acyl group, an alkoxycarbonyl group, a hetero atom-containing cyclic hydrocarbon group, and a trialkylsilyl group include a method of reacting the intermediate and the compound (hereinafter, abbreviated as “protective group introducing agent”) represented by any one of Structural Formulae (5-1) to (5-8):

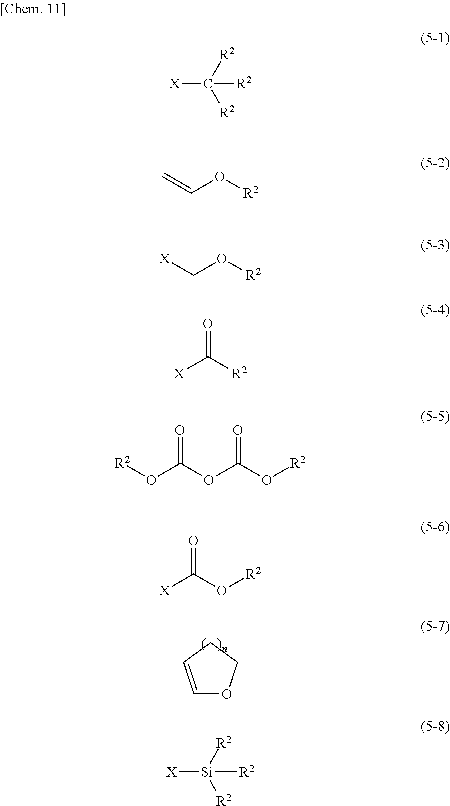

(in the formula, X represents a halogen atom, R²'s each independently represent an alkyl group having 1 to 6 carbon atoms or a phenyl group, and n is 1 or 2).

Among the protective group introducing agents, in view of obtaining the resin in which cleavage under acid catalytic conditions tends to proceed and which is excellent in the photosensitivity, the resolution and the alkali developability, a compound represented by Structural Formula (5-2) or (5-7) is preferable, and ethyl vinyl ether or dihydropyran is particularly preferred.

The method of reacting the intermediate novolac resin with a protective group introducing agent represented by any one of Structural Formulae (5-1) to (5-8) becomes different depending on the compound used as a protective group introducing agent, and in the case where a compound represented by any one of Structural Formulae (5-1), (5-3), (5-4), (5-5), (5-6), and (5-8) is used as the protective group introducing agent, for example, a method of reacting the intermediate novolac resin with the protective group introducing agent under basic catalyst conditions such as pyridine and triethylamine. Further, in the case of using a compound represented by Structural Formula (5-2) or (5-7) as the protective group introducing agent, for example, a method of reacting the intermediate novolac resin and the protective group introducing agent under acidic catalytic conditions such as hydrochloric acid.

The reaction ratio between the intermediate novolac resin and the protective group introducing agent represented by any one of Structural Formulae (5-1) to (5-8) becomes different depending on the compound used as a protective group introducing agent; however, in the structural moiety (X is any one of a hydrogen atom, a tertiary alkyl group, an alkoxyalkyl group, an acyl group, an alkoxycarbonyl group, a hetero atom-containing cyclic hydrocarbon group, and a trialkylsilyl group) represented by —OX present in the obtained novolac resin, the reaction is preferably carried out at a ratio such that the proportion of the structural moiety (OX′) in which X is any one of a tertiary alkyl group, an alkoxyalkyl group, an acyl group, an alkoxycarbonyl group, a hetero atom-containing cyclic hydrocarbon group, and a trialkylsilyl group is 30% to 100%. That is, the reaction is preferably carried out at a ratio such that the protective group introducing agent becomes 0.3 to 1.2 mole, with respect to total 1 mole of the phenolic hydroxyl group in the intermediate novolac resin.

The reaction between the intermediate novolac resin and the protective group introducing agent may be carried out in an organic solvent. Examples of the organic solvent used here include 1,3-dioxolane. Each of these organic solvents may be used singly, or two or more kinds thereof may be used as a mixed solvent.

After the end of the reaction, the desired novolac resin can be obtained, for example, by pouring the reaction mixture into ion-exchanged water and drying the precipitate under reduced pressure.

The novolac resin of the present invention is excellent in the balance between the developability, the heat resistance, and the dry etching resistance, and is suitable for a resist material, and thus preferably contains a dimer in which the number of repeating units of the structural moiety represented by Structural Formula (1) or (2) is 2, or a trimer in which the number of repeating units of the structural moiety represented by Structural Formula (1) or (2) is 3.

Examples of the dimer include those having a molecular structure represented by any one of Structural Formulae (II-1) to (II-3):

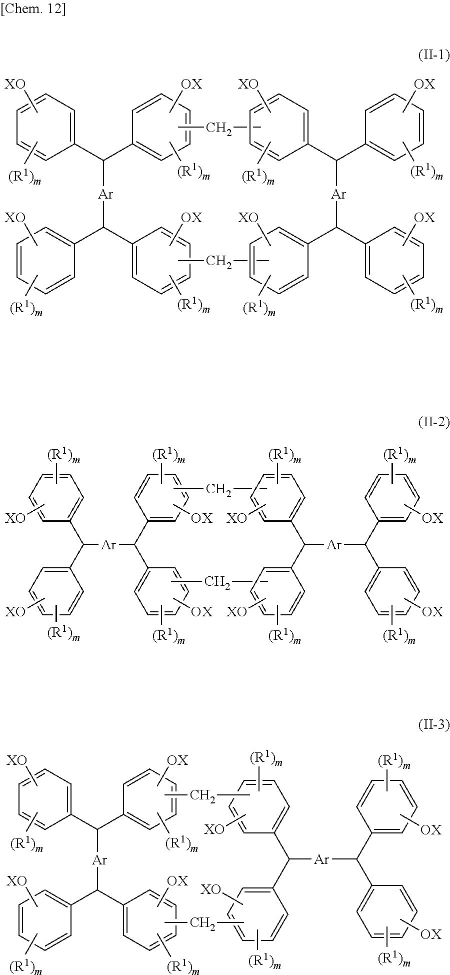

(in the formula, Ar represents an arylene group; R¹'s each independently represent any one of a hydrogen atom, an alkyl group, an alkoxy group, an aryl group, an aralkyl group, and a halogen atom, and m's each independently represent an integer of 1 to 3, and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group).

Examples of the trimer include those having a molecular structure represented by any one of Structural Formulae (III-1) to (III-6):

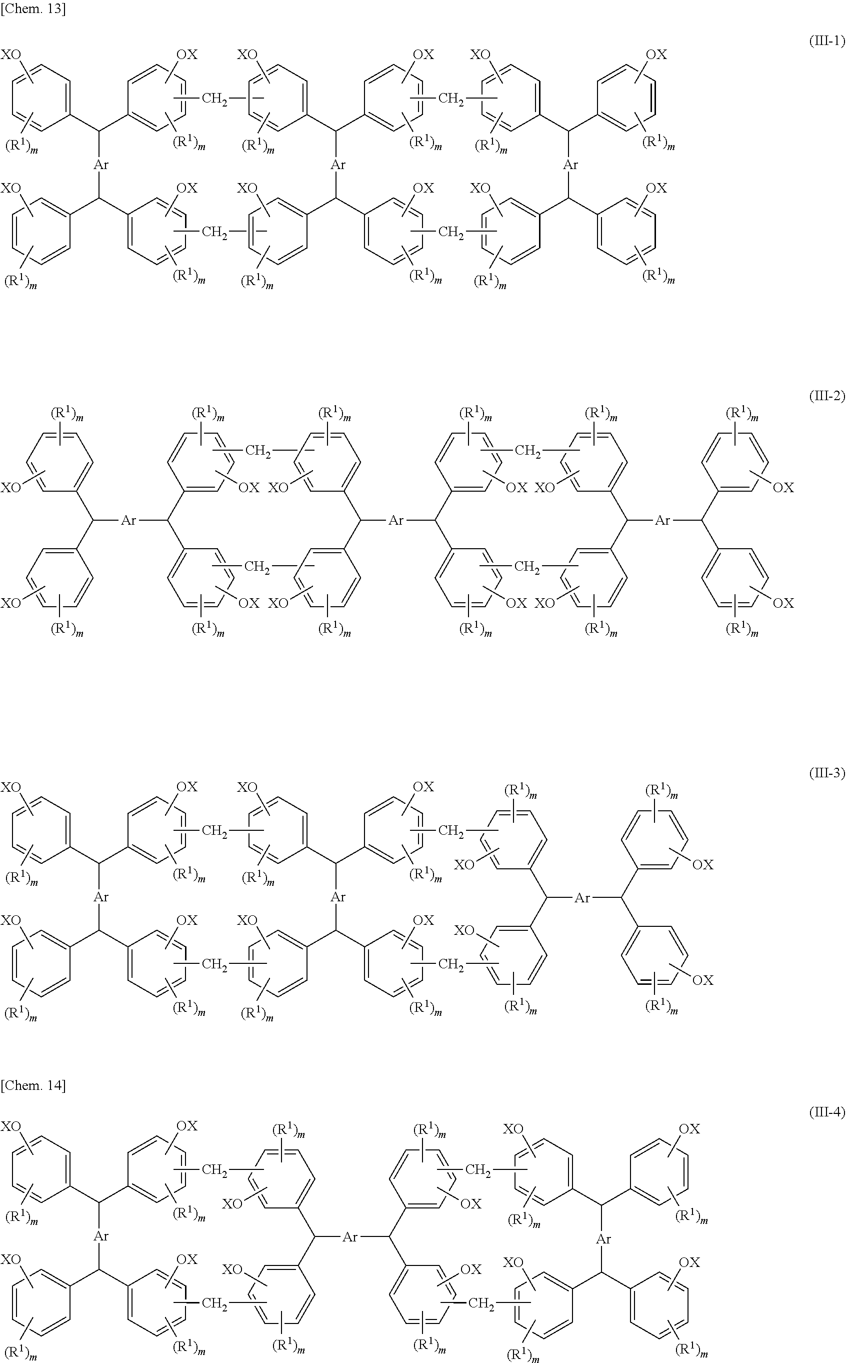

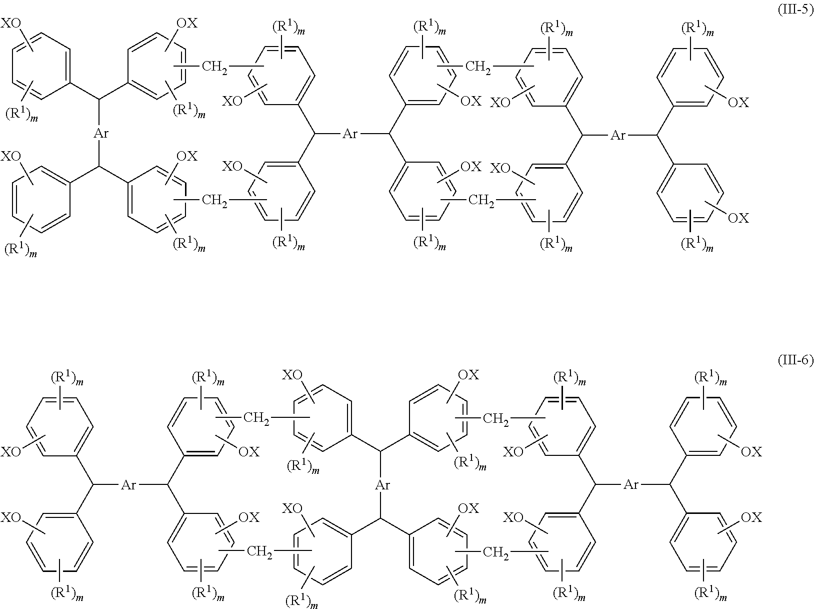

(in the formulae, Ar represents an arylene group; R¹'s each independently represent any one of a hydrogen atom, an alkyl group, an alkoxy group, an aryl group, an aralkyl group, and a halogen atom, and m's each independently represent an integer of 1 to 3, and X is anyone of a hydrogen atom, a tertiary alkyl group, an alkoxyalkyl group, an acyl group, an alkoxycarbonyl group, a hetero atom-containing cyclic hydrocarbon group, and a trialkylsilyl group).

In the case where the novolac resin contains the dimer, the content is particularly preferably 5% to 90% in view of obtaining the novolac resin having excellent developability. In the case where the novolac resin contains the trimer, the content is preferably 5% to 90% in view of obtaining the novolac resin having excellent heat resistance. Note that, the content of the dimer or trimer in the novolac resin is a value calculated from the area ratio of the GPC chart measured under the same conditions as the calculation of the purity of the tetrafunctional phenol compound (A).

The novolac resin of the present invention is easily dissolved in a general-purpose organic solvent and has excellent heat resistance, and thus can be used for various electrical and electronic members such as an adhesive or paint, a photoresist, and a printed wiring board. Among these applications, it is particularly suitable for resist applications that make use of the features of excellent developability, heat resistance and dry etching resistance, and can be used for an alkali developing resist material by being combined with a photosensitive agent, or for a thick film, a resist underlayer film, or a resist permanent film by being combined with a curing agent.

The photosensitive composition of the present invention contains the novolac resin of the present invention and a photoacid generator as essential components.

Examples of the photoacid generator include an organic halogen compound, sulfonic acid ester, an onium salt, a diazonium salt, and a disulfone compound, and each of these may be used alone, or two or more kinds thereof may be used in combination. Specific examples thereof include haloalkyl group-containing s-triazine derivative such as tris(trichloromethyl)-s-triazine, tris(tribromomethyl)-s-triazine, tris(dibromomethyl)-s-triazine, and 2,4-bis(tribromomethyl)-6-p-methoxyphenyl-s-triazine;

a halogen-substituted paraffinic hydrocarbon compound such as 1,2,3,4-tetrabromobutane, 1,1,2,2-tetrabromoethane, carbon tetrabromide, and iodoform; a halogen-substituted cycloparaffinic hydrocarbon compound such as hexabromocyclohexane, hexachlorocyclohexane, and hexabromocyclododecane;

a haloalkyl group-containing benzene derivative such as bis(trichloromethyl) benzene and bis(tribromomethyl) benzene; a haloalkyl group-containing sulfone compound such as tribromomethyl phenyl sulfone and trichloromethyl phenyl sulfone; a halogen-containing sulfolane compounds such as 2,3-dibromosulfolane; a haloalkyl group-containing isocyanurate compound such as tris(2,3-dibromopropyl) isocyanurate;

sulfonium salt such as triphenyl sulfonium chloride, triphenyl sulfonium methanesulfonate, triphenyl sulfonium trifluoromethanesulfonate, diphenyl (4-methylphenyl) sulfonium trifluoromethanesulfonate, triphenyl sulfonium p-toluenesulfonate, triphenylsulfonium tetrafluoroborate, triphenylsulfonium hexafluoroarsenate, and triphenylsulfonium hexafluorophosphonate;

iodonium salt such as diphenyl iodonium trifluoromethanesulfonate, diphenyl iodonium p-toluenesulfonate, diphenyl iodonium tetrafluoroborate, diphenyl iodonium hexafluoroarsenate, and diphenyl iodonium hexafluorophosphonate;

a sulfonic acid ester compound such as methyl p-toluenesulfonate, ethyl p-toluenesulfonate, butyl p-toluenesulfonate, phenyl p-toluenesulfonate, 1,2,3-tris (p-toluenesulfonyloxy) benzene, benzoin p-toluenesulfonate, methyl methanesulfonate, ethyl methanesulfonate, butyl methane sulfonate, 1,2,3-tris (methanesulfonyloxy) benzene, phenyl methanesulfonate, methanesulfonic acid benzoin ester, methyl trifluoromethanesulfonate, ethyl trifluoromethanesulfonate, butyl trifluoromethanesulfonate, 1,2,3-tris (trifluoromethanesulfonyloxy) benzene, phenyl trifluoromethanesulfonate, and tifluoromethanesulfonic acid benzoin ester; disulfone compound such as diphenyl disulfone;

a sulfone diazide compound such as bis(phenyl sulfonyl) diazomethane, bis(2,4-dimethyl phenyl sulfonyl) diazomethane, bis(cyclohexyl sulfonyl) diazomethane, cyclohexyl sulfonyl-(2-methoxy phenyl sulfonyl) diazomethane, cyclohexyl sulfonyl-(3-methoxy phenyl sulfonyl) diazomethane, cyclohexyl sulfonyl-(4-methoxy phenyl sulfonyl) diazomethane, cyclopentyl sulfonyl-(2-methoxy phenyl sulfonyl) diazomethane, cyclopentyl sulfonyl-(3-methoxy phenyl sulfonyl) diazomethane, cyclopentyl sulfonyl-(4-methoxy phenyl sulfonyl) diazomethane, cyclohexyl sulfonyl-(2-fluorophenyl sulfonyl) diazomethane, cyclohexyl sulfonyl-(3-fluorophenyl sulfonyl) diazomethane, cyclohexyl sulfonyl-(4-fluorophenyl sulfonyl) diazomethane, cyclopentyl sulfonyl-(2-fluorophenyl sulfonyl) diazomethane, cyclopentyl sulfonyl-(3-fluorophenyl sulfonyl) diazomethane, cyclopentyl sulfonyl-(4-fluorophenyl sulfonyl) diazomethane, cyclohexyl sulfonyl-(2-chlorophenyl sulfonyl) diazomethane, cyclohexyl sulfonyl-(3-chlorophenyl sulfonyl) diazomethane, cyclohexyl sulfonyl-(4-chlorophenyl sulfonyl) diazomethane, cyclopentyl sulfonyl-(2-chlorophenyl sulfonyl) diazomethane, cyclopentyl sulfonyl-(3-chlorophenyl sulfonyl) diazomethane, cyclopentyl sulfonyl-(4-chlorophenyl sulfonyl) diazomethane, cyclohexyl sulfonyl-(2-trifluoromethyl phenyl sulfonyl) diazomethane, cyclohexyl sulfonyl-(3-trifluoromethyl phenyl sulfonyl) diazomethane, cyclohexyl sulfonyl-(4-trifluoromethyl phenyl sulfonyl) diazomethane, cyclopentyl sulfonyl-(2-trifluoromethyl phenyl sulfonyl) diazomethane, cyclopentyl sulfonyl-(3-trifluoromethyl phenyl sulfonyl) diazomethane, cyclopentyl sulfonyl-(4-trifluoromethyl phenyl sulfonyl) diazomethane, cyclohexyl sulfonyl-(2-trifluoromethoxy phenyl sulfonyl) diazomethane, cyclohexyl sulfonyl-(3-trifluoromethoxy phenyl sulfonyl) diazomethane, cyclohexyl sulfonyl-(4-trifluoromethoxy phenyl sulfonyl) diazomethane, cyclopentyl sulfonyl-(2-trifluoromethoxy phenyl sulfonyl) diazomethane, cyclopentyl sulfonyl-(3-trifluoromethoxy phenyl sulfonyl) diazomethane, cyclopentyl sulfonyl-(4-trifluoromethoxy phenyl sulfonyl) diazomethane, cyclohexyl sulfonyl-(2,4,6-trimethyl phenyl sulfonyl) diazomethane, cyclohexyl sulfonyl-(2,3,4-trimethyl phenyl sulfonyl) diazomethane, cyclohexyl sulfonyl-(2,4,6-triethyl phenyl sulfonyl) diazomethane, cyclohexyl sulfonyl-(2,3,4-triethyl phenyl sulfonyl) diazomethane, cyclopentyl sulfonyl-(2,4,6-trimethyl phenyl sulfonyl) diazomethane, cyclopentyl sulfonyl-(2,3,4-trimethyl phenyl sulfonyl) diazomethane, cyclopentyl sulfonyl-(2,4,6-triethyl phenyl sulfonyl) diazomethane, cyclopentyl sulfonyl-(2,3,4-triethyl phenyl sulfonyl) diazomethane, phenyl sulfonyl-(2-methoxy phenyl sulfonyl) diazomethane, phenyl sulfonyl-(3-methoxy phenyl sulfonyl) diazomethane, phenyl sulfonyl-(4-methoxy phenyl sulfonyl) diazomethane, bis(2-methoxyphenylsulfonyl) diazomethane, bis(3-methoxy phenyl sulfonyl) diazomethane, bis(4-methoxy phenyl sulfonyl) diazomethane, phenyl sulfonyl-(2,4,6-trimethyl phenyl sulfonyl) diazomethane, phenyl sulfonyl-(2,3,4-trimethyl phenyl sulfonyl) diazomethane, phenyl sulfonyl-(2,4,6-triethyl phenyl sulfonyl) diazomethane, phenyl sulfonyl-(2,3,4-triethyl phenyl sulfonyl) diazomethane, 2,4-dimethyl phenyl sulfonyl-(2,4,6-trimethyl phenyl sulfonyl) diazomethane, 2,4-dimethyl phenyl sulfonyl-(2,3,4-trimethyl phenyl sulfonyl) diazomethane, phenyl sulfonyl-(2-fluorophenyl sulfonyl) diazomethane, phenyl sulfonyl-(3-fluorophenyl sulfonyl) diazomethane, and phenyl sulfonyl-(4-fluorophenyl sulfonyl) diazomethane;

an o-nitrobenzyl ester compound such as o-nitrobenzyl-p-toluenesulfonate; and a sulfone hydrazide compound such as N, N′-di(phenylsulfonyl) hydrazide.

The additional amount of these photoacid generators is preferably 0.1 to 20 parts by mass with respect to 100 parts by mass of the resin solid contents of the photosensitive composition in view of obtaining a photosensitive composition with high photosensitivity.

The photosensitive composition of the present invention may contain an organic base compound for neutralizing an acid generated from the photoacid generator at the time of exposure. The addition of the organic base compound has an effect of preventing dimension variation of the resist pattern due to migration of the acid generated from the photoacid generator. As the organic base compound used here, for example, an organic amine compound selected from nitrogen-containing compounds can be mentioned, and specific examples thereof include a pyrimidine compound such as pyrimidine, 2-aminopyrimidine, 4-aminopyrimidine, 5-aminopyrimidine, 2,4-diaminopyrimidine, 2,5-diaminopyrimidine, 4,5-diaminopyrimidine, 4,6-diaminopyrimidine, 2,4,5-triaminopyrimidine, 2,4,6-triaminopyrimidine, 4,5,6-triaminopyrimidine, 2,4,5,6-tetraaminopyrimidine, 2-hydroxy pyrimidine, 4-hydroxy pyrimidine, 5-hydroxy pyrimidine, 2,4-dihydroxy pyrimidine, 2,5-dihydroxy pyrimidine, 4,5-dihydroxy pyrimidine, 4,6-dihydroxy pyrimidine, 2,4,5-trihydroxy pyrimidine, 2,4,6-trihydroxy pyrimidine, 4,5,6-trihydroxy pyrimidine, 2,4,5,6-tetrahydroxy pyrimidine, 2-amino-4-hydroxy pyrimidine, 2-amino-5-hydroxy pyrimidine, 2-amino-4,5-dihydroxy pyrimidine, 2-amino-4,6-dihydroxy pyrimidine, 4-amino-2,5-dihydroxy pyrimidine, 4-amino-2,6-dihydroxy pyrimidine, 2-amino-4-methyl pyrimidine, 2-amino-5-methyl pyrimidine, 2-amino-4,5-dimethyl pyrimidine, 2-amino-4,6-dimethyl pyrimidine, 4-amino-2,5-dimethyl pyrimidine, 4-amino-2,6-dimethyl pyrimidine, 2-amino-4-methoxy pyrimidine, 2-amino-5-methoxy pyrimidine, 2-amino-4,5-dimethoxy pyrimidine, 2-amino-4,6-dimethoxy pyrimidine, 4-amino-2,5-dimethoxy pyrimidine, 4-amino-2,6-dimethoxy pyrimidine, 2-hydroxy-4-methyl pyrimidine, 2-hydroxy-5-methyl pyrimidine, 2-hydroxy-4,5-dimethyl pyrimidine, 2-hydroxy-4,6-dimethyl pyrimidine, 4-hydroxy-2,5-dimethyl pyrimidine, 4-hydroxy-2,6-dimethyl pyrimidine, 2-hydroxy-4-methoxy pyrimidine, 2-hydroxy-4-methoxy pyrimidine, 2-hydroxy-5-methoxy pyrimidine, 2-hydroxy-4,5-dimethoxy pyrimidine, 2-hydroxy-4,6-dimethoxy pyrimidine, 4-hydroxy-2,5-dimethoxy pyrimidine, and 4-hydroxy-2,6-dimethoxy pyrimidine;

a pyridine compound such as pyridine, 4-dimethyl aminopyridine, and 2,6-dimethyl pyridine;

an amine compound substituted with a hydroxyalkyl group having 1 to 4 carbon atoms such as diethanolamine, triethanolamine, triisopropanolamine, tris(hydroxymethyl) aminomethane, and bis(2-hydroxyethyl) iminotris (hydroxymethyl) methane; and

an aminophenol compound such as 2-aminophenol, 3-aminophenol, and 4-aminophenol. Each of these may be used alone, or two or more kinds thereof may be used in combination. Among them, in view of excellent dimensional stability of the resist pattern after the exposure, the pyrimidine compound, a pyridine compound, or an amine compound having a hydroxyl group is preferable, and an amine compound having a hydroxyl group is particularly preferable.

In the case of adding the organic base compound, the additional amount thereof is preferably 0.1% to 100% by mol, and is more preferably 1% to 50% by mol, with respect to the content of the photoacid generator.

The photosensitive composition of the present invention may include other resins (V) in combination with the novolac resin of the present invention. Any of other resins (V) may be used as long as it is soluble in an alkali developing solution or used in combination with an additive such as an acid generating agent to dissolve in the alkali developing solution.

Examples of other resins (V) used here include other phenol resins (V-1) than the novolac resin of the present invention, a homopolymer or copolymer (V-2) of a hydroxyl group-containing styrene compound such as, p-hydroxystyrene and p-(1,1,1,3,3,3-hexafluoro-2-hydroxypropyl) styrene; those (V-3) obtained by modifying the hydroxyl group of (V-1) or (V-2) with an acid-decomposable group such as a t-butoxycarbonyl group or a benzyloxycarbonyl group; a homopolymer or a copolymer (V-4) of (meth) acrylic acid; and an alternating polymer (V-5) of an alicyclic polymerizable monomer such as norbornene compound and tetracyclododecene compound and maleic anhydride or maleimide.

Examples of the other phenol resin (V-1) include phenol resins such as a phenol novolac resin, a cresol novolac resin, a naphthol novolac resin, a co-condensed novolac resin obtained by using various phenolic compounds, an aromatic hydrocarbon formaldehyde resin-modified phenol resin, a dicyclopentadiene phenol adduct resin, a phenol aralkyl resin (XYLOK resin), a naphthol aralkyl resin, a trimethylolmethane resin, a tetraphenylolethane resin, a biphenyl-modified phenol resin (a polyhydric phenol compound in which phenol nuclei are linked by a bismethylene group), a biphenyl-modified naphthol resin (a polyhydric naphthol compound in which phenol nuclei are linked by a bismethylene group), an aminotriazine-modified phenol resin (a polyhydric phenol compound in which phenol nuclei are linked by melamine, benzoguanamine, or the like), and an alkoxy group-containing aromatic ring-modified novolac resin (a polyhydric phenol compound in which a phenol nucleus is linked with an alkoxy group-containing aromatic ring by formaldehyde).

Among the other phenol resins (V-1), in view of obtaining a photosensitive resin composition having high sensitivity and excellent heat resistance, a cresol novolac resin or a co-condensed novolac resin of cresol and another phenolic compound is preferable. The cresol novolac resin or the co-condensed novolac resin of cresol and another phenolic compound is specifically a novolac resin obtained by using at least one cresol selected from the group consisting of o-cresol, m-cresol, and p-cresol and an aldehyde compound as essential raw materials and optionally other suitable phenolic compounds in combination.

Examples of the other phenolic compound than the cresol include phenol; xylenol such as 2,3-xylenol, 2,4-xylenol, 2,5-xylenol, 2,6-xylenol, 3,4-xylenol, and 3,5-xylenol; ethylphenol such as o-ethylphenol, m-ethylphenol, and p-ethylphenol; butylphenol such as isopropylphenol, butylphenol, and p-t-butylphenol; alkylphenol such as p-pentylphenol, p-octylphenol, p-nonylphenol, and p-cumylphenol; halogenated phenol such as fluorophenol, chlorophenol, bromophenol, and iodophenol; monosubstituted phenol such as p-phenylphenol, aminophenol, nitrophenol, dinitrophenol, and trinitrophenol; fused polycyclic phenol such as 1-naphthol and 2-naphthol; and polyhydric phenol such as resorcin, alkyl resorcin, pyrogallol, catechol, alkyl catechol, hydroquinone, alkyl hydroquinone, phloroglucin, bisphenol A, bisphenol F, bisphenol S, and dihydroxynaphthalene. These other phenolic compounds may be used singly, or two or more kinds thereof may be used in combination. In the case where these other phenolic compounds are used, the amount of the compounds used is preferably set such that the number of moles of the other phenolic compounds is 0.05 to 1 mol with respect to the total of 1 mol of the cresol raw material.

Furthermore, examples of the aldehyde compound include formaldehyde, paraformaldehyde, trioxane, acetaldehyde, propionaldehyde, polyoxymethylene, chloral, hexamethylenetetramine, furfural, glyoxal, n-butyl aldehyde, caproaldehyde, allyl aldehyde, benzaldehyde, crotonaldehyde, acrolein, tetraoxymethylene, phenylacetaldehyde, o-tolualdehyde, and salicylaldehyde, and each of these aldehyde compounds may be used singly, or two or more kinds thereof may be used in combination. Among these, in view of excellent reactivity, formaldehyde is preferable, and formaldehyde may be used in combination with another aldehyde compound. In the case where formaldehyde is used in combination with another aldehyde compound, the amount of another aldehyde compound used is preferably 0.05 to 1 mol with respect to 1 mol of formaldehyde.

In view of obtaining the photosensitive resin composition having excellent sensitivity and heat resistance, the reaction ratio between the phenolic compound and the aldehyde compound when producing a novolac resin is set such that the number of moles of the aldehyde compound is preferably 0.3 to 1.6 mol and more preferably 0.5 to 1.3 with respect to 1 mol of the phenolic compound.

Examples of the method for the reaction between the phenolic compound and the aldehyde compound include a method in which the reaction is carried out under the temperature condition of 60° C. to 140° C. in the presence of an acid catalyst and then water and residual monomers are removed under the condition of reduced pressure. Examples of the acid catalyst used here include oxalic acid, sulfuric acid, hydrochloric acid, phenolsulfonic acid, p-toluenesulfonic acid, zinc acetate, and manganese acetate, and each of these acid catalysts may be used singly, or two or more kinds thereof may be used in combination. Among these, from the viewpoint of excellent catalytic activity, oxalic acid is preferable.

Among the cresol novolac resin or the co-condensed novolac resin of cresol and another phenolic compound described above in detail, a cresol novolac resin obtained by solely using m-cresol or a cresol novolac resin obtained by using m-cresol and p-cresol together is preferable. In the latter case, the reaction molar ratio between m-cresol and p-cresol (m-cresol/p-cresol) is preferably 10/0 to 2/8 and more preferably 7/3 to 2/8, in view of obtaining the photosensitive resin composition having excellent balance between sensitivity and heat resistance.

In the case where the other resin (V) is used, the blending ratio between the novolac resin of the present invention and the other resin (V) can be optionally adjusted according to the desired use. For example, since optical sensitivity, resolution, and heat resistance of the novolac resin of the present invention are excellent when the resin is used in combination with the photosensitizing agent, the photosensitive composition including the novolac resin of the present invention and the photosensitizing agent as the main components is optimal for use in a resist. Here, in view of obtaining a curable composition having high optical sensitivity and excellent resolution and heat resistance, the proportion of the novolac resin of the present invention in the total resin components is preferably 60% by mass or higher and more preferably 80% by mass or higher.

The novolac resin of the present invention can be used as a sensitivity improving agent by making use of the characteristic of excellent optical sensitivity of the resin. In this case, the blending ratio between the novolac resin and the other resin (V) is preferably set such that the amount of the novolac resin of the present invention is 3 to 80 parts by mass with respect to 100 parts by mass of the other resin (V).

The photosensitive composition of the present invention may include a photosensitizing agent which is used for ordinary resist materials. Examples of the photosensitizing agent include a compound having a quinone diazide group. Specific examples of the compound having a quinone diazide group include a complete ester compound, a partial ester compound, an amidated product, or a partial amidated product of an aromatic (poly)hydroxy compound and sulfonic acid having a quinone diazide group such as naphthoquinone-1,2-diazide-5-sulfonic acid, naphthoquinone-1,2-diazide-4-sulfonic acid, and ortho-anthraquinone diazide sulfonic acid.

Examples of the aromatic (poly)hydroxy compound used here include a polyhydroxybenzophenone compound such as 2,3,4-trihydroxybenzophenone, 2,4,4′-trihydroxybenzophenone, 2,4,6-trihydroxybenzophenone, 2,3,6-trihydroxybenzophenone, 2,3,4-trihydroxy-2′-methylbenzophenone, 2,3,4,4′-tetrahydroxybenzophenone, 2,2′,4,4′-tetrahydroxybenzophenone, 2,3′,4,4′,6-pentahydroxybenzophenone, 2,2′,3,4,4′-pentahydroxybenzophenone, 2,2′,3,4,5-pentahydroxybenzophenone, 2,3′,4,4′,5′,6-hexahydroxybenzophenone, and 2,3,3′,4,4′,5′-hexahydroxybenzophenone;

a bis[(poly)hydroxyphenyl]alkane compound such as bis(2,4-dihydroxyphenyl)methane, bis(2,3,4-trihydroxyphenyl)methane, 2-(4-hydroxyphenyl)-2-(4′-hydroxyphenyl)propane, 2-(2,4-dihydroxyphenyl)-2-(2′,4′-dihydroxyphenyl)propane, 2-(2,3,4-trihydroxyphenyl)-2-(2′,3′,4′-trihydroxyphenyl)propane, 4,4′-{1-[4-[2-(4-hydroxyphenyl)-2-propyl]phenyl]ethylidene}bisphenol, and 3,3′-dimethyl-{1-[4-[2-(3-methyl-4-hydroxyphenyl)-2-propyl]phenyl]ethylidene}bisphenol;

a tris(hydroxyphenyl)methane compound such as tris(4-hydroxyphenyl)methane, bis(4-hydroxy-3,5-dimethylphenyl)-4-hydroxyphenylmethane, bis(4-hydroxy-2,5-dimethylphenyl)-4-hydroxyphenylmethane, bis(4-hydroxy-3,5-dimethylphenyl)-2-hydroxyphenylmethane, bis(4-hydroxy-2,5-dimethylphenyl)-2-hydroxyphenylmethane, bis(4-hydroxy-2,5-dimethylphenyl)-3,4-dihydroxyphenylmethane, and bis(4-hydroxy-3,5-dimethylphenyl)-3,4-dihydroxyphenylmethane or a methyl substitution product thereof;

and a bis(cyclohexylhydroxyphenyl)(hydroxyphenyl)methane compound such as bis(3-cyclohexyl-4-hydroxyphenyl)-3-hydroxyphenylmethane, bis(3-cyclohexyl-4-hydroxyphenyl)-2-hydroxyphenylmethane, bis(3-cyclohexyl-4-hydroxyphenyl)-4-hydroxyphenylmethane, bis(5-cyclohexyl-4-hydroxy-2-methylphenyl)-2-hydroxyphenyl methane, bis(5-cyclohexyl-4-hydroxy-2-methylphenyl)-3-hydroxyphenyl methane, bis(5-cyclohexyl-4-hydroxy-2-methylphenyl)-4-hydroxyphenyl methane, bis(3-cyclohexyl-2-hydroxyphenyl)-3-hydroxyphenylmethane, bis(5-cyclohexyl-4-hydroxy-3-methylphenyl)-4-hydroxyphenyl methane, bis(5-cyclohexyl-4-hydroxy-3-methylphenyl)-3-hydroxyphenyl methane, bis(5-cyclohexyl-4-hydroxy-3-methylphenyl)-2-hydroxyphenyl methane, bis(3-cyclohexyl-2-hydroxyphenyl)-4-hydroxyphenylmethane, bis(3-cyclohexyl-2-hydroxyphenyl)-2-hydroxyphenylmethane, bis(5-cyclohexyl-2-hydroxy-4-methylphenyl)-2-hydroxyphenyl methane, and bis(5-cyclohexyl-2-hydroxy-4-methylphenyl)-4-hydroxyphenyl methane or a methyl substitution product thereof. Each of these photosensitizing agents may be used singly, or two or more kinds thereof may be used in combination.

In view of obtaining the photosensitive composition having excellent optical sensitivity, the blending amount of the photosensitizing agent in the photosensitive composition of the present invention is preferably 5 to 50 parts by mass with respect to 100 parts by mass of the total of the resin solid contents in the photosensitive composition.

The photosensitive composition of the present invention may include a surfactant, for example, for the purpose of improving film forming properties and adhesiveness of a pattern and reducing development defects in the case of using the composition for a resist. Examples of the surfactant used here include a nonionic surfactant such as a polyoxyethylene alkyl ether compound such as polyoxyethylene lauryl ether, polyoxyethylene stearyl ether, polyoxyethylene cetyl ether, and polyoxyethylene oleyl ether, a polyoxyethylene alkyl allyl ether compound such as polyoxyethylene octylphenol ether, and polyoxyethylene nonylphenol ether, a sorbitan fatty acid ester compound such as polyoxyethylene⋅polyoxypropylene block copolymer, sorbitan monolaurate, sorbitan monopalmitate, sorbitan monostearate, sorbitan monooleate, sorbitan trioleate, and sorbitan tristearate, and a polyoxyethylene sorbitan fatty acid ester compound such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate; a fluorine-based surfactant having a fluorine atom in the molecular structure thereof such as a copolymer of a polymerizable monomer having a fluoroaliphatic group and [poly(oxyalkylene)] (meth)acrylate; and a silicone-based surfactant having a silicone structural moiety in the molecular structure thereof. These surfactants may be used singly, or two or more kinds thereof may be used in combination.

The blending amount of the surfactant is preferably 0.001 to 2 parts by mass with respect to 100 parts by mass of the total of the resin solid contents in the photosensitive composition of the present invention.

In the case where the photosensitive composition of the present invention is used for a photoresist, the composition can be used as a composition for a resist by adding the novolac resin of the present invention, the photoacid generator, and, as necessary, the other phenol resin (V), a sensitizing agent, and various additives such as a surfactant, a dye, a filler, a crosslinking agent, and a dissolution promotor, and dissolving the above components in an organic solvent. This may be used as a positive tone resist solution as it is, or the composition may be utilized as a positive tone resist film formed by applying the composition in a film shape and removing the solvent. Examples of a support film when used as the resist film include a synthetic resin film such as polyethylene, polypropylene, polycarbonate, and polyethylene terephthalate, and the film may be used as a single layer film or a plurality of multilayer films. The surface of the support film may be subjected to a corona treatment or may be coated with a release agent.

The organic solvent used for the composition for a resist of the present invention is not particularly limited, and examples thereof include alkylene glycol monoalkyl ether such as ethylene glycol monomethyl ether, ethylene glycol monoethyl ether, ethylene glycol monopropyl ether, ethylene glycol monobutyl ether, and propylene glycol monomethyl ether; dialkylene glycol dialkyl ether such as diethylene glycol dimethyl ether, diethylene glycol diethyl ether, diethylene glycol dipropyl ether, and diethylene glycol dibutyl ether; alkylene glycol alkyl ether acetate such as ethylene glycol monomethyl ether acetate, ethylene glycol monoethyl ether acetate, and propylene glycol monomethyl ether acetate; a ketone compound such as acetone, methyl ethyl ketone, cyclohexanone, and methyl amyl ketone; a cyclic ether such as dioxane; and an ester compound such as methyl 2-hydroxypropionate, ethyl 2-hydroxypropionate, ethyl 2-hydroxy-2-methylpropionate, ethyl ethoxyacetate, ethyl oxyacetate, methyl 2-hydroxy-3-methylbutanoate, 3-methoxybutyl acetate, 3-methyl-3-methoxybutyl acetate, ethyl formate, ethyl acetate, butyl acetate, methyl acetoacetate, and ethyl acetoacetate. Each of these organic solvents may be used singly, or two or more kinds thereof may be used in combination.

The composition for a resist of the present invention can be prepared by blending each of the components and mixing with a stirrer or the like. In the case where a resin composition for a photoresist includes a filler or a pigment, the composition can be prepared by dispersing or mixing the components with a dispersing device such as a dissolver, a homogenizer, and a three roll mill.

In a photolithography method using the composition for a resist of the present invention, for example, an object to be subjected to photolithography, such as silicon substrate, is coated with the composition for a resist, and prebaking is performed under a temperature condition of 60° C. to 150° C. A coating method used here may be any method such as spin coating, roll coating, flow coating, dip coating, spray coating, and doctor blade coating. Next, a resist pattern is created, however, since the composition for a resist of the present invention is a positive-tone resist composition, a resist pattern is formed by performing exposure through a prescribed mask to provide a desired resist pattern and dissolving the exposed portion with an alkali developer. In the composition for a resist of the present invention, both alkali solubility of the exposed portion and alkali insolubility of the unexposed portion are high, and thus, it is possible to form a resist pattern with excellent resolution.

The curable composition of the present invention includes the novolac resin of the present invention and a curing agent as essential components. In the curable composition of the present invention, a resin (W) other than the novolac resin of the present invention may be used together with the above components. Examples of the other resin (W) used here include various novolac resins, a resin formed by addition polymerization of an alicyclic diene compound such as dicyclopentadiene and a phenol compound, a modified novolac resin of a phenolic hydroxyl group-containing compound and an alkoxy group-containing aromatic compound, a phenol aralkyl resin (XYLOK resin), a naphthol aralkyl resin, a trimethylolmethane resin, a tetraphenylolethane resin, a biphenyl-modified phenol resin, a biphenyl-modified naphthol resin, an aminotriazine-modified phenol resin, and various vinyl polymers.

More specifically, examples of the various novolac resins include a polymer obtained by reacting a phenolic hydroxyl group-containing compound, for example, phenol, alkylphenol such as cresol and xylenol, phenylphenol, resorcinol, biphenyl, bisphenol such as bisphenol A and bisphenol F, naphthol, and dihydroxynaphthalene with an aldehyde compound, under the condition of an acid catalyst.

Examples of the various vinyl polymer include a homopolymer or a copolymer of vinyl compounds such as polyhydroxystyrene, polystyrene, polyvinyl naphthalene, polyvinyl anthracene, polyvinyl carbazole, polyindene, polyacenaphthylene, polynorbornene, polycyclodecene, polytetracyclododecene, polynortricyclene, and poly(meth)acrylate.

In the case where these other resins are used, the blending ratio between the novolac resin of the present invention and the other resin (W) can be optionally set according to the use, however, in view of more remarkably expressing the effect of excellent dry etching resistance and resistance to thermal decomposition exhibited by the present invention, the blending ratio is preferably set such that the amount of the other resin (W) is 0.5 to 100 parts by mass with respect to 100 parts by mass of the novolac resin of the present invention.

Examples of the curing agent used in the present invention include a melamine compound substituted with at least one group selected from the group consisting of a methylol group, an alkoxymethyl group, and an acyloxymethyl group, a guanamine compound, a glycoluril compound, a urea compound, a resole resin, an epoxy compound, an isocyanate compound, an azide compound, a compound containing a double bond such as an alkenyl ether group, an acid anhydride, and an oxazoline compound.

Examples of the melamine compound include hexamethylol melamine, hexamethoxymethyl melamine, a compound in which one to six methylol groups of hexamethylol melamine are methoxy methylated, hexamethoxyethyl melamine, hexaacyloxymethyl melamine, and a compound in which one to six methylol groups of hexamethylol melamine are acyloxymethylated.

Examples of the guanamine compound include tetramethylol guanamine, tetramethoxymethyl guanamine, tetramethoxymethyl benzoguanamine, a compound in which one to four methylol groups of tetramethylol guanamine are methoxy methylated, tetramethoxyethyl guanamine, tetraacyloxy guanamine, and a compound in which one to four methylol groups of tetramethylol guanamine are acyloxymethylated.

Examples of the glycoluril compound include 1,3,4,6-tetrakis(methoxymethyl)glycoluril, 1,3,4,6-tetrakis(butoxymethyl)glycoluril, and 1,3,4,6-tetrakis(hydroxymethyl)glycoluril.

Examples of the urea compound include 1,3-bis(hydroxymethyl)urea, 1,1,3,3-tetrakis(butoxymethyl)urea, and 1,1,3,3-tetrakis(methoxymethyl)urea.

Examples of the resol resin include a polymer obtained by reacting a phenolic hydroxyl group-containing compound, for example, phenol, alkylphenol such as cresol and xylenol, phenylphenol, resorcinol, biphenyl, bisphenol such as bisphenol A and bisphenol F, naphthol, and dihydroxynaphthalene with an aldehyde compound under the condition of an alkali catalyst.

Examples of the epoxy compound include diglycidyloxynaphthalene, a phenol novolac-type epoxy resin, a cresol novolac-type epoxy resin, a naphthol novolac-type epoxy resin, a naphthol-phenol co-condensed novolac-type epoxy resin, a naphthol-cresol co-condensed novolac-type epoxy resin, a phenol aralkyl-type epoxy resin, a naphthol aralkyl-type epoxy resin, 1,1-bis(2,7-diglycidyloxy-1-naphthyl)alkane, a naphthylene ether-type epoxy resin, a triphenyl methane-type epoxy resin, a dicyclopentadiene-phenol addition reaction-type epoxy resin, a phosphorus atom-containing epoxy resin, and a polyglycidyl ether of a co-condensate of a phenolic hydroxyl group-containing compound and an alkoxy group-containing aromatic compound.

Examples of the isocyanate compound include tolylene diisocyanate, diphenylmethane diisocyanate, hexamethylene diisocyanate, and cyclohexane diisocyanate.

Examples of the azide compound include 1,1′-biphenyl-4,4′-bis azide, 4,4′-methylidene bis azide, and 4,4′-oxy bis azide.

Examples of the compound containing a double bond such as an alkenyl ether group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

Examples of the acid anhydride include an aromatic acid anhydride such as phthalic anhydride, trimellitic anhydride, pyromellitic anhydride, 3,3′,4,4′-benzophenonetetracarboxylic dianhydride, biphenyltetracarboxylic dianhydride, 4,4′-(isopropylidene)diphthalic anhydride, and 4,4′-(hexafluoroisopropylidene)diphthalic anhydride; and an alicyclic carboxylic anhydride such as tetrahydrophthalic anhydride, methyltetrahydrophthalic anhydride, hexahydrophthalic anhydride, methylhexahydrophthalic anhydride, endomethylenetetrahydrophthalic anhydride, dodecenylsuccinic anhydride, and trialkyltetrahydrophthalic anhydride.

Among these, in view of obtaining the curable composition having excellent curability and heat resistance in the cured product, a glycoluril compound, a urea compound, and a resol resin are preferable, and a glycoluril compound is particularly preferable.

In view of obtaining the composition having excellent curability, the blending amount of the curing agent in the curable composition of the present invention is preferably 0.5 to 50 parts by mass with respect to 100 parts by mass of the total of the novolac resin of the present invention and the other resin (W).

In the case where the curable composition of the present invention is used for a resist underlayer film (BARC film), a composition for a resist underlayer film can be prepared by adding the novolac resin of the present invention, the curing agent, and as necessary, the other resin (W) and various additives such as a surfactant, a dye, a filler, a crosslinking agent, and a dissolution promotor, and dissolving the above components in an organic solvent.

The organic solvent used for the composition for a resist underlayer film is not particularly limited, and examples thereof include alkylene glycol monoalkyl ether such as ethylene glycol monomethyl ether, ethylene glycol monoethyl ether, ethylene glycol monopropyl ether, ethylene glycol monobutyl ether, and propylene glycol monomethyl ether; dialkylene glycol dialkyl ether such as diethylene glycol dimethyl ether, diethylene glycol diethyl ether, diethylene glycol dipropyl ether, and diethylene glycol dibutyl ether; alkylene glycol alkyl ether acetate such as ethylene glycol monomethyl ether acetate, ethylene glycol monoethyl ether acetate, and propylene glycol monomethyl ether acetate; a ketone compound such as acetone, methyl ethyl ketone, cyclohexanone, and methyl amyl ketone; a cyclic ether such as dioxane; and an ester compound such as methyl 2-hydroxypropionate, ethyl 2-hydroxypropionate, ethyl 2-hydroxy-2-methylpropionate, ethyl ethoxyacetate, ethyl oxyacetate, methyl 2-hydroxy-3-methylbutanoate, 3-methoxybutyl acetate, 3-methyl-3-methoxybutyl acetate, ethyl formate, ethyl acetate, butyl acetate, methyl acetoacetate, and ethyl acetoacetate. Each of these organic solvents may be used singly, or two or more kinds thereof may be used in combination.

The composition for a resist underlayer film can be prepared by blending each of the components and performing mixing with a stirrer or the like. In the case where the composition for a resist underlayer film includes a filler or a pigment, the composition can be prepared by dispersing or mixing the components with a dispersing device such as a dissolver, a homogenizer, and a three roll mill.

In the case where the resist underlayer film is prepared from the composition for a resist underlayer film, the resist underlayer film is formed by a method in which an object to be subjected to photolithography such as a silicon substrate is coated with the composition for a resist underlayer film, and the composition is dried under the temperature condition of 100° C. to 200° C., and then thermally cured under the temperature condition of 250° C. to 400° C. Next, a resist pattern can be formed using a multilayer resist method by performing a conventional photolithography operation on the underlayer film so as to form a resist pattern and performing a dry etching treatment using a halogen-based plasma gas or the like.

In the case where the curable composition of the present invention is used for a resist permanent film, a composition for a resist permanent film can be prepared by adding the novolac resin of the present invention, the curing agent, and as necessary, the other phenol resin (W) and various additives such as a surfactant, a dye, a filler, a crosslinking agent, and a dissolution promotor, and dissolving the above components in an organic solvent. Examples of the organic solvent used here are the same as the examples of the organic solvents used for the composition for a resist underlayer film.

In a photolithography method using the composition for a resist permanent film, for example, the resin component and the additive components are dissolved and dispersed in the organic solvent and then applied onto an object to be subjected to photolithography, such as silicon substrate, and prebaking is performed under a temperature condition of 60° C. to 150° C. A coating method used here may be any method such as spin coating, roll coating, flow coating, dip coating, spray coating, and doctor blade coating. Next, a resist pattern is created, however, in the case where the composition for a resist permanent film is a positive-tone composition, a resist pattern is formed by performing exposure through a prescribed mask to provide a desired resist pattern and dissolving the exposed portion with an alkali developer.

In the case of a semiconductor device, for example, a permanent film formed of the composition for a resist permanent film can be suitably used in a packaging adhesive layer for a solder resist, a packaging material, an underfill material, and a circuit element, or an adhesive layer between an integrated circuit element and a circuit substrate, and in the case of a thin display represented by LCD and OELD, the permanent film formed from the composition for a resist permanent film can be suitably used in a thin film transistor protective film, a liquid crystal color filter protective film, a black matrix, or a spacer.

### EXAMPLES

The present invention will be described in more detail using the following specific examples. The number average molecular weight (Mn), the weight average molecular weight (Mw), and the polydispersity (Mw/Mn) of a synthesized resin were measured by GPC under the following measurement condition. The purity and the contents of dimer or trimer were calculated based on the area ratio in the GPC chart obtained under the following measurement condition.

[Measurement condition for GPC]

Measurement device: “HLC-8220 GPC” manufactured by TOSOH CORPORATION

Column: “Shodex KF802” (8.0 mmϕ×300 mm) manufactured by SHOWA DENKO K.K.+“Shodex KF802” (8.0 mmϕ×300 mm) manufactured by SHOWA DENKO K.K.

+“Shodex KF803” (8.0 mmϕ×300 mm) manufactured by SHOWA DENKO K.K.+“Shodex KF804” (8.0 mmϕ×300 mm) manufactured by SHOWA DENKO K.K.

Column temperature: 40° C.

Detector: RI (differential refractometer)

Data processing: “GPC-8020 MODEL II VERSION 4.30” manufactured by TOSOH CORPORATION

Eluent: tetrahydrofuran

Flow rate: 1.0 mL/min

Sample: a sample obtained by filtering 0.5% by mass (in terms of a resin solid content) of tetrahydrofuran solution through a microfilter

Injection volume: 0.1 mL

Standard sample: the following monodisperse polystyrene

(Standard sample: monodisperse polystyrene)

“A-500” manufactured by TOSOH CORPORATION

“A-2500” manufactured by TOSOH CORPORATION

“A-5000” manufactured by TOSOH CORPORATION

“F-1” manufactured by TOSOH CORPORATION

“F-2” manufactured by TOSOH CORPORATION

“F-4” manufactured by TOSOH CORPORATION

“F-10” manufactured by TOSOH CORPORATION

“F-20” manufactured by TOSOH CORPORATION

For the measurement of the ¹H-NMR spectrum, structural analysis was carried out by analyzing the sample DMSO-d₆ solution using “AL-400” manufactured by JEOL Ltd. Measurement conditions of ¹H-NMR spectrum are shown below.

[Measurement conditions for ¹H-NMR spectrum]

Measurement mode: SGNNE (1H complete decoupling method of NOE elimination)

Pulse angle: 45° C. pulse

Sample concentration: 30 wt %

Cumulative number: 10000 times

For the measurement of the ¹³C-NMR spectrum, structural analysis was carried out by analyzing the sample DMSO-d₆ solution using “AL-400” manufactured by JEOL Ltd. The measurement condition for the ¹³C-NMR spectrum is shown below.

[Measurement condition for ¹³C-NMR spectrum]

Measurement mode: SGNNE (1H complete decoupling method of NOE elimination)

Pulse angle: 45° C. pulse

Sample concentration: 30 wt %

Cumulative number: 10,000 times

In measurement of the TOF-MS spectrum, the sample was analyzed using “AXIMA TOF2” manufactured by Shimadzu Corporation and using dithranol as a matrix and sodium trifluoroacetate as a cationization agent, so as to perform molecular weight analysis.

Measurement mode: linear mode

Sample adjustment: sample/dithranol/sodium trifluoroacetate/THF=10/10/1/1

### Production Example 1

Production of Tetrafunctional Phenol Compound (A-1)

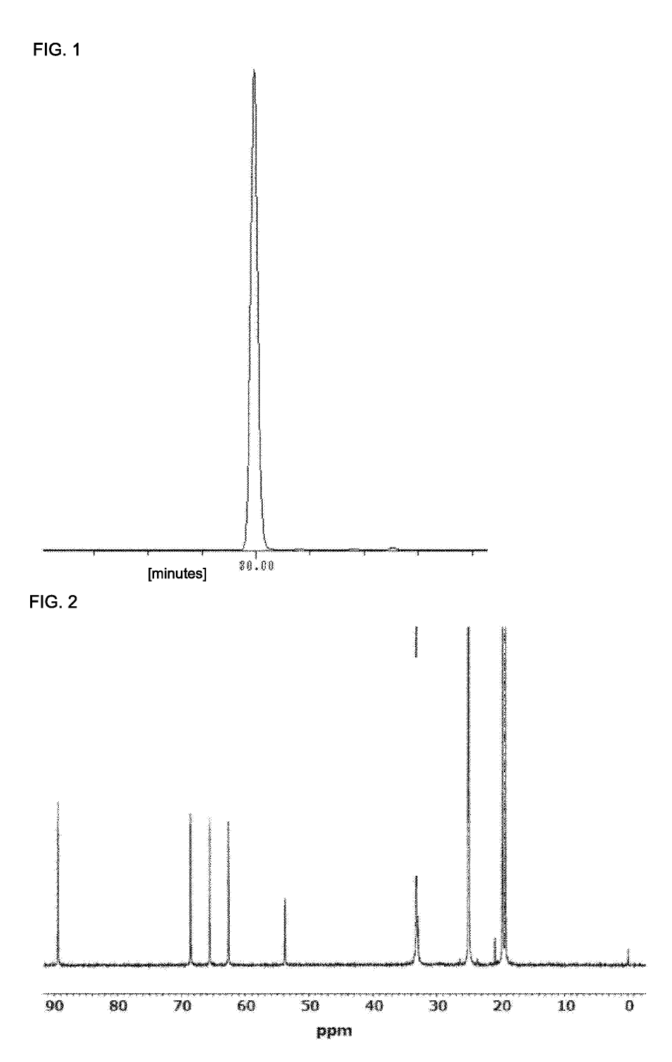

A 100 ml two-neck flask equipped with a cooling tube was charged with 73 g (0.6 mol) of 2,5-xylenol and 20 g (0.15 mol) of terephthalaldehyde, which were then dissolved in 300 ml of 2-ethoxyethanol. While being cooled in an ice bath, 10 g of sulfuric acid was added thereto, and then heating at 80° C. in an oil bath and stirring were performed for two hours, thereby allowing a reaction to take place. After the reaction, water was added to the obtained solution, and the crude product was reprecipitated. The precipitated crude product was redissolved in acetone and further reprecipitated in water. The precipitate was then isolated by filtration, and vacuum drying was performed, thereby obtaining 62 g of a light red powder of tetrafunctional phenol compound (A-1). The generation of a compound represented by the following structural formula was confirmed by ¹H-NMR. In addition, the purity calculated from a GPC chart diagram was 98.2%. The GPC chart of the tetrafunctional phenol compound (A-1) is shown in FIG. 1, and the ¹H-NMR chart is shown in FIG. 2.

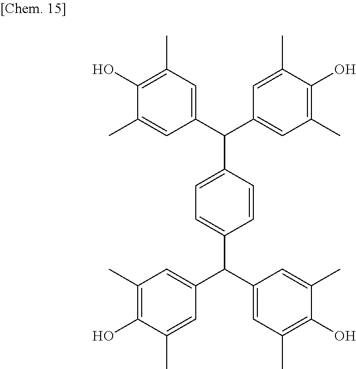 [SMILES: Cc1cc(C(c2ccc(C(c3cc(C)c(O)c(C)c3)c3cc(C)c(O)c(C)c3)cc2)c2cc(C)c(O)c(C)c2)cc(C)c1O]

### Production Example 2

Production of Intermediate Novolac Resins (1) and (2)

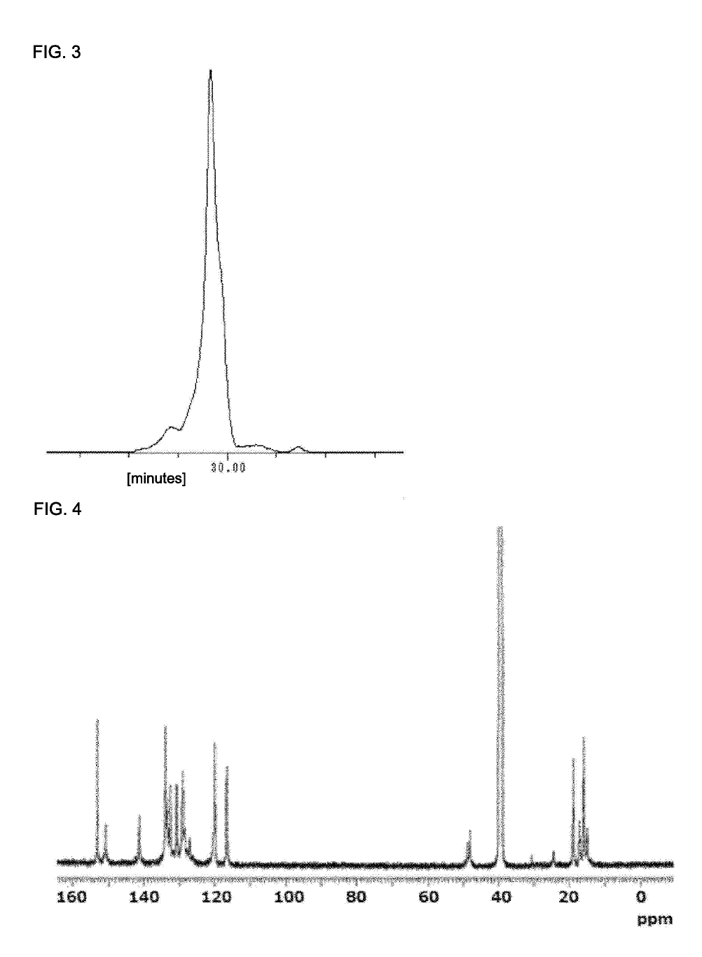

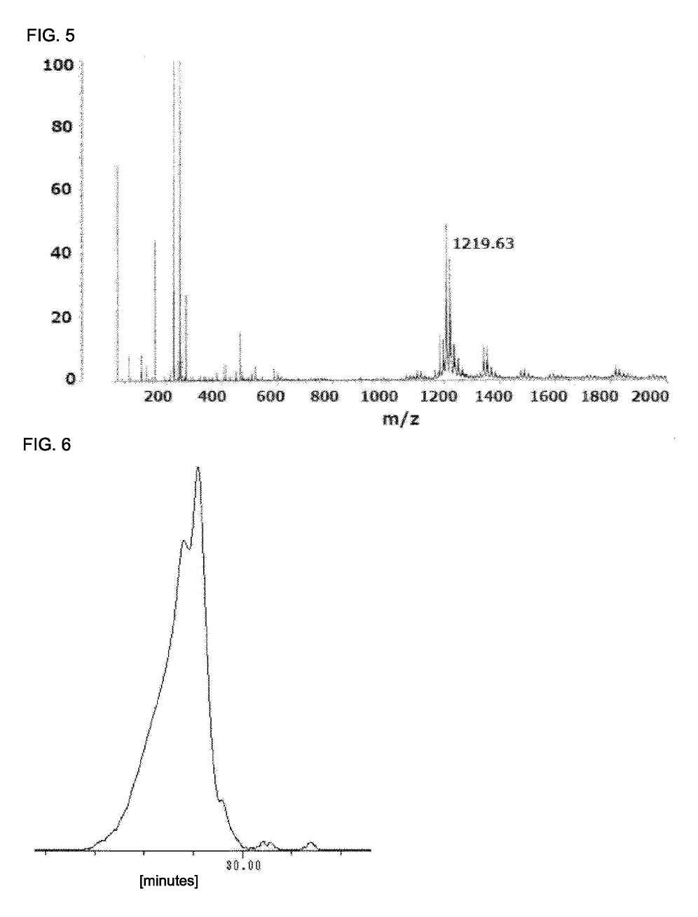

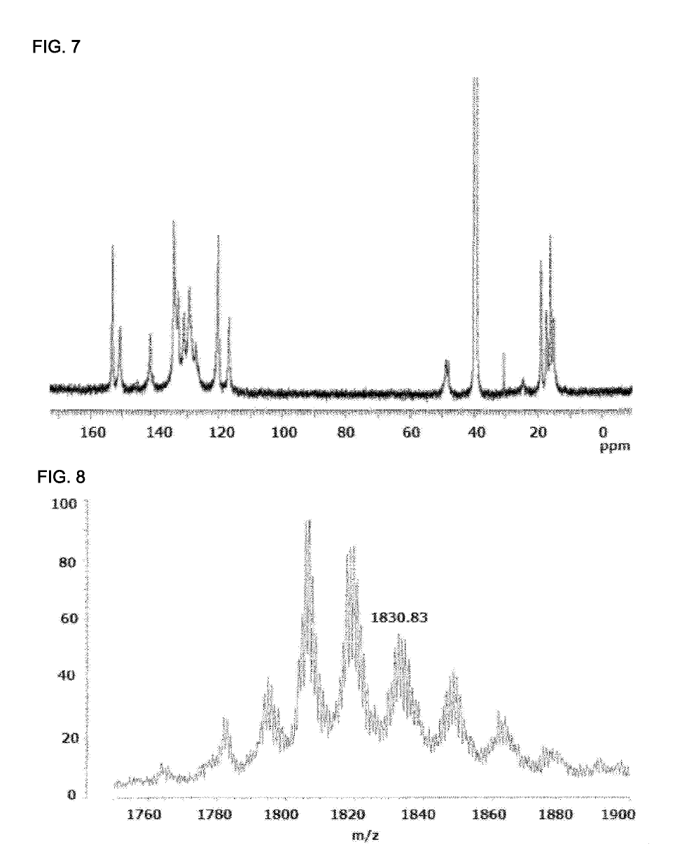

In a 2 L four-necked flask equipped with a cooling tube, 59 g (0.1 mol) of tetrafunctional phenol compound (A-1) obtained in Production Example 1 was dissolved in a mixed solution of 250 ml of methanol and 250 ml of acetic acid. While being cooled in an ice bath, 20 g of sulfuric acid was added thereto, and then 15 g (0.5 mol) of 92% paraformaldehyde was charged and the temperature was raised to 60° C. in a water bath. After heating and stirring were continued for 10 hours to allow the reaction to proceed, water was added to the obtained solution to precipitate the product, which was filtered and dried under vacuum to obtain a crude product of a red solid. The crude product was purified in a silica gel column (eluent: hexane/ethyl acetate=1/1), obtaining 23.4 g of an intermediate novolac resin (1) including a dimer as a main component and 21.6 g of an intermediate novolac resin (2) including a trimer as a main component. The GPC, ¹³C-NMR, and TOF-MS of the intermediate novolac resin (1) are shown in FIG. 3, FIG. 4, and FIG. 5, respectively, and the GPC, ¹³C-NMR, and TOF-MS of the intermediate novolac resin (2) are shown in FIG. 6, FIG. 7, and FIG. 8, respectively. The intermediate novolac resin (1) had a number average molecular weight (Mn) of 1,552, a weight average molecular weight (Mw) of 1,666, and a polydispersity index (Mw/Mn) of 1.07, and a peak of 1,219 indicating the presence of a dimeric sodium adduct was observed in the TOF-MS spectrum. The intermediate novolac resin (2) had a number average molecular weight (Mn) of 2,832, a weight average molecular weight (Mw) of 3,447, and a polydispersity index (Mw/Mn) of 1.22, and a peak of 1,830 indicating the presence of a trimeric sodium adduct was observed in the TOF-MS spectrum.

### Example 1

Production of Novolac Resin (1)

A 100 ml three-necked flask equipped with a cooling tube was charged with 6 g of intermediate novolac resin (1) synthesized in Production Example 2, and 4 g of ethyl vinyl ether as a protective group introducing agent, which were then dissolved in 30 g of 1,3-dioxolane. After adding 0.01 g of a 35 wt % hydrochloric acid aqueous solution, stirring was continued at 25° C. for four hours for reaction. Titration was carried out with methanol during the reaction, and after confirming a methanol dissolved component disappeared and the protective group was introduced into almost all of the hydroxyl groups, 0.1 g of a 25 wt % aqueous ammonia solution was added. Water was added to the obtained solution to perform a reprecipitation operation, and the precipitate was filtered and dried under vacuum to obtain 6.2 g of a red powder of novolac resin (1).

### Example 2

Production of Novolac Resin (2)

6.7 g of a red powder of novolac resin (2) was obtained by performing the same operation as in Example 1 except that as a protective group introducing agent, 4.4 g of dihydropyran was used instead of 4 g of ethyl vinyl ether.

### Example 3

Production of Novolac Resin (3)

6.1 g of a red powder of novolac resin (3) was obtained by performing the same operation as in Example 1 except that 6 g of intermediate novolac resin (2) was used instead of 6 g of intermediate novolac resin (1).

### Example 4

Production of Novolac Resin (4)

6.4 g of a red powder of novolac resin (4) was obtained by performing the same operation as in Example 3 except that 6 g of intermediate novolac resin (2) was used as the phenol resin before protection and 4.4 g of dihydropyran, as a protective group introducing agent, was used instead of 4 g of ethyl vinyl ether.

### Comparative Production Example 1

Production of Novolac Resin (1′)

A 2 L four-necked flask equipped with a stirrer and a thermometer was charged with 648 g (6 mol) of m-cresol, 432 g (4 mol) of p-cresol, 2.5 g (0.2 mol) of oxalic acid, and 492 g of 42% formaldehyde, and the temperature was raised to 100° C., thereby allowing a reaction to take place. Dehydration and distillation were performed under the conditions of normal pressure and a temperature of 200° C. and distillation under reduced pressure was performed at 230° C. for six hours to obtain 736 g of a light yellow solid of intermediate novolac resin (1′). The intermediate novolac resin (1′) had a number average molecular weight (Mn) of 1,450, a weight average molecular weight (Mw) of 10,316, and a polydispersity index (Mw/Mn) of 7.116.

6.7 g of a novolac resin (1′) was obtained by performing the same operation as in Example 2 except that 6 g of intermediate novolac resin (1′) was used instead of 6 g of intermediate novolac resin (1).

### Examples 5 to 8 and Comparative Example 1

For each of the novolac resins obtained in Examples 1 to 5 and Comparative Production Example 1, a photosensitive composition was prepared in the following manner and various evaluations were carried out. The results are shown in Table 1.

Preparation of Photosensitive Composition

1.9 g of novolac resin was dissolved in 8 g of propylene glycol monomethyl ether acetate, and 0.1 g of photoacid generator was added to the solution and dissolved. This solution was filtered through a 0.2 μm membrane filter, thereby obtaining a photosensitive composition.

“WPAG-336” [diphenyl (4-methyl phenyl) sulfonium trifluoromethanesulfonate] manufactured by Wako Pure Chemical Industries, Ltd. was used as a photoacid generator.

Preparation of Composition for Testing Heat Resistance

1.9 g of novolac resin was dissolved in 8 g of propylene glycol monomethyl ether acetate, and this solution was filtered through a 0.2 μm membrane filter, thereby obtaining a composition for testing heat resistance.

Evaluation of Alkali Developability [ADR (Nm/s)]

A 5-inch silicon wafer was coated with the photosensitive composition obtained above with a spin coater such that the thickness of the composition became approximately 1 μm, and the composition was dried on a hot plate at 110° C. for 60 seconds. Two wafers were prepared in such way, and one was designated as a “sample without exposure”. The other one was used as an “exposed sample” and was irradiated with a ghi line at 100 mJ/cm² using a ghi line lamp (“MULTILIGHT” manufactured by USHIO INC.) and was subjected to a heating treatment at 140° C. for 60 seconds.

Both of the “sample without exposure” and the “exposed sample” were immersed in an alkali developer (2.38% tetramethylammonium hydroxide aqueous solution) for 60 seconds, and then the samples were dried on a hot plate at 110° C. for 60 seconds. Film thicknesses of each sample before and after the immersion in the developer were measured, and a value obtained by dividing the difference in the thickness by 60 was designated as alkali developability [ADR (nm/s)].

Evaluation of Optical Sensitivity

A 5-inch silicon wafer was coated with the photosensitive composition obtained above with a spin coater such that the thickness of the composition became approximately 1 μm, and the composition was dried on a hot plate at 110° C. for 60 seconds. A mask corresponding to a resist pattern in which the line and space was 1:1 and a line width was set within 1 to 10 μm in increments of 1 μm was adhered onto the wafer, the wafer was irradiated with a ghi line using a ghi line lamp (“MULTILIGHT” manufactured by USHIO INC.), and then subjected to a heating treatment at 140° C. for 60 seconds. Next, the wafer was immersed in an alkali developer (2.38% tetramethylammonium hydroxide aqueous solution) for 60 seconds and dried on a hot plate at 110° C. for 60 seconds.

In the case where the exposure amount of the ghi line was increased from 30 mJ/cm² in increments of 5 mJ/cm², an exposure amount (Eop exposure amount) at which a line width of 3 μm was able to be faithfully reproduced was evaluated.

Evaluation of Resolution

A 5-inch silicon wafer was coated with the photosensitive composition obtained above with a spin coater such that the thickness of the composition became approximately 1 μm, and the composition was dried on a hot plate at 110° C. for 60 seconds. A photomask was placed on the obtained wafer, the wafer was irradiated with a ghi line at 200 mJ/cm², using the same method as in the case of the evaluation of alkali developability above, and an alkali developing operation was performed. A state of a pattern was confirmed using a laser microscope (“VK-X200” manufactured by KEYENCE CORPORATION.), and a pattern that was able to be resolved at L/S=5 μm was evaluated as A, and a pattern that was not able to be resolved at L/S=5 μm was evaluated as B.

Evaluation of Heat Resistance

A 5-inch silicon wafer was coated with the composition for testing heat resistance obtained above with a spin coater such that the thickness of the composition became approximately 1 μm, and the composition was dried on a hot plate at 110° C. for 60 seconds. A resin was scraped off from the obtained wafer, and a glass transition temperature (Tg) of the resin was measured. The glass transition temperature (Tg) was measured using a differential scanning calorimeter (DSC) (“Q100” manufactured by TA Instruments) under a nitrogen atmosphere and under the condition of a temperature range of −100° C. to 200° C. and a temperature rising at a rate of 10° C./min.

TABLE 1

| | | | | | | Comparative |
|---|---|---|---|---|---|---|
| | | Example 5 | Example 6 | Example 7 | Example 8 | Example 1 |
| Novolac resin | | (1) | (2) | (3) | (4) | (1′) |
| Alkali | “Sample | 0 | 0 | 0 | 0 | 0 |
| developability | without | | | | | |
| ADR (nm/s) | exposure” | | | | | |
| | “Exposed | >700 | >700 | 620 | 560 | 21 |
| | sample” | | | | | |

| | | | | | |
|---|---|---|---|---|---|
| Optical sensitivity | 25 | 25 | 30 | 30 | 230 |
| [mJ/cm²] | | | | | |
| Resolution | A | A | A | A | B |
| Heat resistance [° C.] | 231 | 235 | 249 | 253 | 86 |

### Examples 9 to 12, and Comparative Example 2

For the novolac resin obtained in Examples 1 to 4 and Comparative Production Example 1, a curable composition was prepared in the following manner and various evaluation tests were carried out. The results are indicated in Table 2.

Preparation of Curable Composition

1.6 g of novolac resin and 0.4 g of a curing agent (“1,3,4,6-tetrakis (methoxymethyl) glycoluril” manufactured by Tokyo Chemical Industry Co., Ltd.) were dissolved in 3 g of propylene glycol monomethyl ether acetate, and this solution was filtered through a 0.2 μm membrane filter, thereby obtaining a curable composition.

Evaluation of Dry Etching Resistance

A 5-inch silicon wafer was coated with the curable composition obtained above with a spin coater, and the composition was dried on a hot plate at 110° C. for 60 seconds. Heating was performed in a hot plate, in which the oxygen concentration was 20% by volume, at 180° C. for 60 seconds, and heating was further performed at 350° C. for 120 seconds, thereby obtaining a silicon wafer with a cured coating film having a film thickness of 0.3 μm. An etching treatment was performed on the cured coating film on the wafer using an etching unit (“EXAM” manufactured by SHINKO SEIKI CO., LTD.) under the condition of CF₄/Ar/O₂ (CF₄: 40 mL/min, Ar: 20 mL/min, O₂: 5 mL/min; pressure: 20 Pa; RF power: 200 W; treatment time: 40 seconds; temperature: 15° C.). Film thicknesses before and after the etching treatment were measured at this time, the etching rate was calculated, and the etching resistance was evaluated. The evaluation criteria are as below.

A: the case where an etching rate is 150 nm/min or lower

B: the case where an etching rate exceeds 150 nm/min

TABLE 2

| | Example | Example | Example | Example | Comparative |
|---|---|---|---|---|---|
| | 9 | 10 | 11 | 12 | Example 2 |
| Novolac resin | (1) | (2) | (3) | (4) | (1′) |
| Dry etching | A | A | A | A | B |
| resistance | | | | | |

1. A novolac resin comprising, as a repeating unit,
a structural moiety represented by Structural Formula (1) or (2): 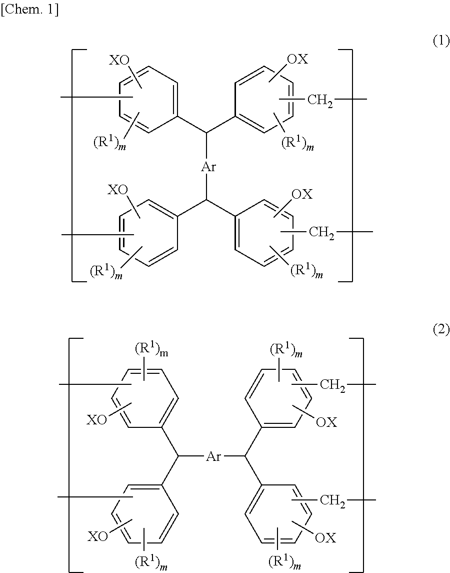
(wherein Ar represents an arylene group, R¹'s each independently represent any one of a hydrogen atom, an alkyl group, an alkoxy group, and a halogen atom, m's each independently represent an integer of 1 to 3, and X is any one of a hydrogen atom, a tertiary alkyl group, an alkoxyalkyl group, an acyl group, an alkoxycarbonyl group, a hetero atom-containing cyclic hydrocarbon group, and a trialkylsilyl group),
wherein at least one of X's present in the resin is any one of a tertiary alkyl group, an alkoxyalkyl group, an acyl group, an alkoxycarbonyl group, a hetero atom-containing cyclic hydrocarbon group, and a trialkylsilyl group.

2. The novolac resin according to claim 1, comprising:
a dimer in which the number of repeating units of the structural moiety represented by Structural Formula (1) or (2) is 2, or a trimer in which the number of repeating units of the structural moiety represented by Structural Formula (1) or (2) is 3.

3. A composition comprising the novolac resin according to claim 1.

4. A resist material comprising the composition according to claim 3.

5-8. (canceled)

9. A cured product, which is formed by curing the composition according to claim 3.

10. A composition comprising the novolac resin according to claim 2.

11. A resist material comprising the composition according to claim 10.

12. A cured product, which is formed by curing the composition according to claim 10.
